SEQUENCE LISTING (20 sequences)

SEQ ID NO: 1 - protein, 6 aa, Artificial Sequence
  Anti-thrombin peptide
GLDPRX

SEQ ID NO: 2 - protein, 6 aa, Artificial Sequence
  Anti-thrombin peptide
GVDPRX

SEQ ID NO: 3 - protein, 6 aa, Artificial Sequence
  Anti-thrombin peptide
GFNPRX

SEQ ID NO: 4 - protein, 6 aa, Artificial Sequence
  Anti-thrombin peptide
GPNPRX

SEQ ID NO: 5 - protein, 6 aa, Artificial Sequence
  Anti-thrombin peptide
GFSARX

SEQ ID NO: 6 - protein, 5 aa, Artificial Sequence
  Anti-thrombin peptide
GVSPR

SEQ ID NO: 7 - protein, 5 aa, Artificial Sequence
  Anti-thrombin peptide
GLDPR

SEQ ID NO: 8 - protein, 5 aa, Artificial Sequence
  Anti-thrombin peptide
GVDPR

SEQ ID NO: 9 - protein, 5 aa, Artificial Sequence
  Anti-thrombin peptide
GFNPR

SEQ ID NO: 10 - protein, 5 aa, Artificial Sequence
  Anti-thrombin peptide
GPNPR

SEQ ID NO: 11 - protein, 5 aa, Artificial Sequence
  Anti-thrombin peptide
GFSAR

SEQ ID NO: 12 - protein, 13 aa, Artificial Sequence
  Anti-thrombin peptide
GDYEEIPEEYLQX

SEQ ID NO: 13 - protein, 13 aa, Artificial Sequence
  Anti-thrombin peptide
GDLEEIPEEYLQX

SEQ ID NO: 14 - protein, 12 aa, Artificial Sequence
  Anti-thrombin peptide
GDGEEIPEEYLQ

SEQ ID NO: 15 - protein, 11 aa, Artificial Sequence
  Anti-thrombin peptide
DFEEIPEEYLQ

SEQ ID NO: 16 - protein, 35 aa, Artificial Sequence
  six Anti-thrombin peptides in tandem
GLDPRMGVDPRMGFNPRMGPNPRMGFSARMGVSPR

SEQ ID NO: 17 - protein, 4 aa, Artificial Sequence
  Anti-thrombin peptide
FDPR

SEQ ID NO: 18 - protein, 18 aa, Artificial Sequence
  Anti-thrombin peptide
PQSHNDGDFEEIPEEYLQ

SEQ ID NO: 19 - protein, 12 aa, Artificial Sequence
  Anti-thrombin peptide
GDFEEIPEEYLQ

SEQ ID NO: 20 - protein, 22 aa, Artificial Sequence
  Anti-thrombin peptide
FDPRPQSHNDGDFEEIPEEYLQ

(19) United States
(12) Patent Application Publication
(10) Pub. No.: US 2005/0287527 A1
(43) Pub. Date: Dec. 29, 2005

(54) Quantitative ranking of transient ligand binding to target biomolecules

(75) Inventors: Feng Ni, Pierrefonds, QC (CA); Zhengding Su, St. Laurent (CA); Ping Xu, Montreal (CA); Dmitri Tolkatchev, Montreal (CA); Michael J. Osborne, Montreal (CA); Anatol Koutychenko, Montreal (CA)

Correspondence Address: NATIONAL RESEARCH COUNCIL OF CANADA;1500 MONTREAL ROAD, BLDG M-58, ROOM EG12, OTTAWA, ONTARIO, K1A 0R6, CA

(73) Assignee: National Research Council of Canada

(21) Appl. No.: 10/500,878

(22) PCT Filed: Jan. 10, 2003
(86) PCT No.: PCT/CA03/00014

§ 371 Date: Jun. 17, 2005

Related U.S. Application Data
(60) Provisional application No. 60/346,894, filed on Jan. 11, 2002.

(51) Int. Cl. C12Q 1/68; G01N 33/00

(52) U.S. Cl. 435/6; 436/86

(57) ABSTRACT

There is provided a method of quantitatively ranking transient ligand binding to target biomolecules by means of NMR relaxation dispersion profiles. The present invention also relates to a method to identify ligand site obeying two-state and more complex binding behavior in a transient complex of a ligand with a target molecule, still with the use of NMR. There is also provided an efficient method to quantitate fast dissociation rates of ligands containing at least one magnetic nuclei by performing NMR relaxation dispersion experiments at different protein concentrations, enabling the evaluation of populations and exchange rates, and extending the practical applicability of the NMR relaxation dispersion experiments.

### TECHNICAL FIELD

The present invention relates to a new use of NMR for quantitatively ranking transient ligand binding to target biomolecules.

### BACKGROUND OF THE INVENTION

The molecular nature of protein-ligand associations is a subject of tremendous interest in chemistry, biochemistry and pharmaceutical drug discovery research (Kuntz, I. D., Chen, K., Sharp, K. A., and Kollman, P. A. (1999) Proc.Natl.Acad.Sci.U.S.A 96, 9997-10002; and Brooijmans, N., Sharp, K. A., and Kuntz, I. D. (2002) Proteins 48, 645-653). Targeting enzymes and protein surfaces involved in molecular regulatory pathways by low-molecular-weight molecules leads to various means of maintaining human health and treating diseases. Thermodynamic analyses of complex formation, such as equilibrium binding experiments and enzyme inhibition assays, have provided valuable information regarding the molecular/atomic forces that dictate the structural stability of protein-ligand complexes (Kuntz, I. D., Chen, K., Sharp, K. A., and Kollman, P. A. (1999) Proc.Natl.Acad.Sci.U.S.A 96, 9997-10002; and Brooijmans, N., Sharp, K. A., and Kuntz, I. D. (2002) Proteins 48, 645-653). In general, however, it is still difficult if not impossible to explain quantitatively the exact correlations between molecular structures and the binding affinity of protein-ligand complexes (Kuntz, I. D., Chen, K., Sharp, K. A., and Kollman, P. A. (1999) Proc.Natl.Acad.Sci.U.S.A 96, 9997-10002). In most cases, the difficulties are related to the failure to account for even subtle molecular motions in the otherwise very exact dimensions of microscopic protein-ligand interactions captured by high-resolution molecular structures (Kuntz, I. D., Chen, K., Sharp, K. A., and Kollman, P. A. (1999) Proc.Natl.Acad.Sci.U.S.A 96, 9997-10002; and Nienaber, V. L., Mersinger, L. J., and Kettner, C. A. (1996) Biochemistry 35, 9690-9699; and Carlson, H. A. (2002) Curr.Opin.Chem.Biol. 6, 447-452). On top of all these, there is as yet no clear understanding of the relationship between binding, kinetics and molecular structure of protein-ligand interactions (Van Regenmortel, M. H. (2001) Cell Mol.Life Sci. 58, 794-800; and Andersson, K., Choulier, L., Hamalainen, M. D., Van Regenmortel, M. H., Altschuh, D., and Malmqvist, M. (2001) J.Mol.Recognit. 14, 62-71; and Day, Y. S., Baird, C. L., Rich, R. L., and Myszka, D. G. (2002) Protein Sci. 11, 1017-1025). The significance of protein-ligand binding kinetics becomes particularly high in vivo, where transient complexes are formed and broken as a cause or in response to biomolecular regulatory mechanisms.

A number of techniques are already available for quantitating the kinetics of protein-protein and protein-ligand interactions, such as surface plasmon resonance (Van Regenmortel, M. H. (2001) Cell Mol.Life Sci. 58, 794-800; and Wilson, W. D. (2002) Science 295, 2103-2105) and analysis of the progress curves of enzyme inhibition by specific ligands (Pargellis, C. A., Morelock, M. M., Graham, E. T., Kinkade, P., Pav, S., Lubbe, K., Lamarre, D., and Anderson, P. C. (1994) Biochemistry 33, 12527-12534; and Day, Y. S., Baird, C. L., Rich,. R. L., and Myszka, D. G. (2002) Protein Sci. 11, 1017-1025). All these methodologies are limited to the characterization of tight-binding or slow-dissociating protein-ligand complexes with lifetimes longer than at least several seconds (Van Regenmortel, M. H. (2001) Cell Mol.Life Sci. 58, 794-800; and Day, Y. S., Baird, C. L., Rich, R. L., and Myszka, D. G. (2002) Protein Sci. 11, 1017-1025). Another limitation of these methods is that they only provide a macroscopic description of binding kinetics, without details of the dynamic behavior of the interacting molecules at atomic resolution.

Short-lived or transient, but specific-binding, protein-ligand complexes represent a good starting point for the design of high affinity inhibitors or effectors (Wells, J. A. (1996) Science 273, 449450; and Shuker, S. B., Hajduk, P. J., Meadows, R. P., and Fesik, S. W. (1996) Science 274, 1531-1534). These fast dissociating ligands are often derived from naturally occurring protein-protein interfaces (Song, J. and Ni, F. (1998) Biochem.Cell Biol. 76, 177-188) or discovered by screening against peptide and/or protein libraries (Wells, J. A. (1996) Science 273, 449450; and Mourez, M., Kane, R. S., Mogridge, J., Metallo, S., Deschatelets, P., Sellman, B. R., Whitesides, G. M., and Collier, R. J. (2001) Nat.Biotechnol. 19, 958-961). Even without affinity “maturation”, these specific-binding ligands can be converted into bivalent and polyvalent molecules with a dramatic increase in affinity and decrease in the dissociation rates (Song, J. and Ni, F. (1998) Biochem.Cell Biol. 76, 177-188; and Mammen, M., Choi, S.-K., and Whitesides, G. M. (1998) Angew.Chem.Int.Ed. 37, 2754-2794; and Rao, J., Lahiri, J., Isaacs, L., Weis, R. M., and Whitesides, G. M. (1998) Science 280, 708-711; and Kramer, R. H. and Karpen, J. W. (1998) Nature 395, 710-713; and Kitov, P. I., Sadowska, J. M., Mulvey, G., Armstrong, G. D., Ling, H., Pannu, N. S., Read, R. J., and Bundle, D. R. (2000) Nature 403, 669-672; and Fan, E., Zhang, Z., Minke, W. E., Hou, Z., Verlinde, C. L. M. J., and Hol, W. G. J. (2000) J.Am.Chem.Soc. 122, 2663-2664; and Kiessling, L. L., Gestwicki, J. E., and Strong, L. E. (2000) Curr.Opin.Chem.Biol. 4, 696-703; and Mourez, M., Kane, R. S., Mogridge, J., Metallo, S., Deschatelets, P., Sellman, B. R., Whitesides, G. M., and Collier, R. J. (2001) Nat.Biotechnol. 19, 958-961).

Nuclear magnetic resonance (NMR) spectroscopy has been established as one of the most powerful tools for studying the kinetic processes in chemical systems. Fast chemical interconversions often lead to extensive broadening of the NMR signals, from which the underlining rate constants and energetic parameters can be derived (Sandstrom, J. (1982) Dynamic NMR spectroscopy. London: Academic Press; and Blackledge, M. J., Bruschweiler, R., Griesinger, C., Schmidt, J. M., Xu, P., and Ernst, R. R. (1993) Biochemistry 32, 10960-10974). In contrast, fast exchange processes in biological systems have very rarely been measured in the details needed for an adequate understanding of the underlying kinetic processes. The binding kinetics of large and transient enzyme-inhibitor and protein-ligand complexes were only estimated by use of NMR spectroscopy and other methods (Jardetzky, O. and Roberts, G. C. K. (1981) NMR in molecular biology. New York: Academic Press; and Hammes, G. G. (1982) Enzyme catalysis and regulation. New York: Academic Press). Some earlier attempts at quantitative measurements utilized mostly ¹⁹F NMR, including spin-lock T₁ₚ), T₁/T₂ and T₂ (CPMG) relaxation experiments (Sykes, B. D. (1969) J.Am.Chem.Soc. 91, 949-955; and Smallcombe, S. H., Ault, B., and Richards, J. H. (1972).J.Am.Chem.Soc. 94, 4585-4590; and Gerig, J. T. and Stock, A. D. (1975) Org.Magn.Res. 7, 249-255; and Gerig, J. T., Halley, B. A., and Ortiz, C. E. (1977) J.Am.Chem.Soc. 99, 6219-6226; and Dubois, B. W. and Evers, A. S. (1992) Biochemistry 31, 7069-7076) and spectral lineshape analysis (Jacobson, A. R. and Gerig, J. T. (1991) J.Biomol.NMR 1, 131-144). More recent developments include combined analyses of proton T₁, T₁ₚ, T₂ and T₂ (CPMG) relaxation data (Davis, D. G., Perlman, M. E., and London, R. E. (1994) J.Magn Reson.B 104, 266-275) or the use of ¹⁹F cross-correlated relaxation measurements (Peng, J. W. (2001) J.Magn Reson. 153, 32-47) to help deconvolute the relaxation dispersion curves of the ligand molecules. The NMR T₂ (CPMG) relaxation measurements in particular appeared to be a promising technique for quantitating the binding kinetics of rapidly-dissociating protein-ligand complexes (Carver, J. P. and Richards, R. E. (1972) J.Magn.Reson. 6, 89-105; and Gerig, J. T. and Stock, A. D. (1975) Org.Magn.Res. 7, 249-255; and Gerig, J. T., Halley, B. A., and Ortiz, C. E. (1977) J.Am.Chem.Soc. 99, 6219-6226; and Dubois, B. W. and Evers, A. S. (1992) Biochemistry 31, 7069-7076; and Davis, D. G., Perlman, M. E., and London, R. E. (1994) J.Magn Reson.B 104, 266-275).

The kinetics of transient protein-ligand interactions has been investigated recently in a few cases, one for a small protein-peptide complex (Hensmann, M., Booker, G. W., Panayotou, G., Boyd, J., Linacre, J., Waterfield, M., and Campbell, I. D. (1994) Protein Sci. 3, 1020-1030; and Gunther, U., Mittag, T., Schaffhausen, and B. (2002) Biochemistry 41, 11658-11669), and some for enzyme-substrate/inhibitor interactions (Deng, H., Zhadin, N., and Callender, R. (2001) Biochemistry 40, 3767-3773; and Gulotta, M., Deng, H., Deng, H., Dyer, R. B., and Callender, R. H. (2002) Biochemistry 41, 3353-3363; and Hammes, G. G. (2002) Biochemistry 41, 8221-8228). The studied protein-peptide complex in particular involves binding of a small protein (MW˜12 kDa) with a phosphotyrosine-containing peptide with equilibrium dissociation constant in the 30-100 nM range (Hensmann, M., Booker, G. W., Panayotou, G., Boyd, J., Linacre, J., Waterfield, M., and Campbell, I. D. (1994) Protein Sci. 3, 1020-1030). To characterize binding kinetics of the protein-peptide complex, the protein was recombinantly expressed, enriched uniformly with the ¹⁵N isotope, and the NMR peak shapes in response to peptide titrations were analyzed. As a practical alternative, it is particularly attractive to deconvolute the kinetic contributions to protein-ligand interactions by following the NMR spectroscopic and relaxation behaviors of the small ligand molecules. This approach expands the capability of quantitating dissociation kinetics to systems where the binding proteins and other biomolecules are large or not perfectly folded, and therefore are not normally or easily accessible to direct NMR observation. In addition, the target proteins do not have to be isotopically labeled and therefore can be purified from the natural sources or from a wide range of recombinant expression systems. Small ligand molecules require considerably less amount of time for NMR signal identification, and can be observed and resolved spectroscopically when mixed with other molecules with comparable molecular weights. The challenge here lies in the difficulty or impossibility to observe the bound states of the ligands due to the high molecular weight of the protein-ligand complex, such that conclusions may depend on the kinetic mechanism chosen to explain the experimental data.

### SUMMARY OF THE INVENTION

One aim of the present invention is to provide a new use of the dissociation rates (k_{off}) of protein-ligand complexes as a measure of the potency of ligand molecules binding transiently to target proteins and other biomolecules.

Another aim of the present invention is to provide an efficient method for quantitating the fast dissociation rates of transient protein-ligand complexes with lifetimes ranging from a few milliseconds to hundreds of microseconds.

In accordance with the present invention there is provided an efficient method to quantitate fast dissociation rates of ligands containing one or more, and preferably at least two, magnetic nuclei by performing NMR relaxation dispersion experiments at different protein concentrations, enabling the evaluation of populations and exchange rates, and extending the practical applicability of the NMR relaxation dispersion experiments.

Nuclear magnetic resonance (NMR)-based tools have been developed for quantitating the binding kinetics of transient protein-ligand complexes. More specifically, it is shown herein that implementation of NMR relaxation dispersion spectroscopy in accordance with the present invention can be used to determine the dissociation rate constants or binding off-rates of protein-peptide complexes in the absence of an accurate knowledge of the concentrations of either the peptide or the binding protein.

It was found and detailed herein that the problem of discriminating two-state from multistate binding processes can be resolved by performing NMR relaxation dispersion experiments of only ligand NMR signals at a number of concentrations of the binding protein.

In accordance with the present invention, there is therefore provided a method to identify two or more ligand molecules that can be linked together to create high-affinity molecules. Specifically, it is proposed herein that bivalent or polyvalent molecules with enhanced binding capacity can be constructed from ligand molecules that bind non-competitively or cooperatively. Non-competitivity or positive coooperativity is to be measured by an unchanged or a decreased dissociation rate constant, k_{off}, of one ligand in the presence of other ligand molecules.

In accordance with the present invention, there is therefore provided a method to identify binding hotspots of high-affinity ligand-target interactions through chemical fragmentation of the ligand molecule.

In accordance with the present invention, there is provided a method to identify a ligand site obeying a two-state or more complex binding behavior in a transient complex of a ligand with a target molecule, said method comprising the steps of:
a) preparing a ligand with at least one nucleus, and more preferably at least two nuclei, detectable by NMR;
b) collecting CPMG NMR relaxation dispersion profiles for free ligand at two or more magnetic fields;
c) determining apparent transverse relaxation rates for the nuclei detectable by NMR at two or more magnetic fields;
d) assigning resonance peaks to the specific NMR detectable nuclei of the ligand with one- and/or multi-dimensional NMR;
e) contacting the ligand with at least one, preferably at least two, and more preferably three concentrations of a target molecule;
f) collecting CPMG NMR relaxation dispersion profiles for the ligand with every concentration of the target molecule at two or more magnetic fields;
g) fitting the NMR relaxation dispersion profiles by a two-state exchange model independently for every nucleus, and independently or simultaneously for every concentration of the target molecule; and
h) determining a ligand site obeying a two-state binding behavior based on feasibility of extracted R_{2b} and p_{b} parameters or the quality of the fitting of step g).

In accordance with the present invention there is provided a method to determine quantitatively the dissociation rate constant (k_{off}) for a transient complex of a ligand with the target molecule comprising the steps of:
a) Identifying a ligand site obeying a two-state or more complex binding behavior in a transient complex of a ligand with a target molecule with the method as defined above; and
b) Extracting k_{off} values for the ligand sites obeying two-site or more complex exchange mechanism, said off values being a measure of the affinity of a transient complex of the ligand with the target molecule.

The ligand may be for example, without limitation a peptide, a ¹⁵N-enriched polypeptide, or a molecule binding to the target under study. The ligand may also be a mixture of any of the above

The target may be for example, without limitation a protein or a protein assembly.

In accordance with the present invention, there is provided the use of the method as defined above to determine amino acid residues with detectable NMR relaxation dispersion as a constituting binding hot-spot.

The method of the present invention may also be used to identify two or more ligands that can be linked together to create high-affinity molecules.

The method of the present invention may still be used to study high-affinity protein-protein interactions or slow-dissociating ligand-target complexes.

It is also proposed that structure-affinity relationships of protein-ligand complexes can be built using the dissociation rate constants alone instead of the commonly used binding equilibrium constants.

### BRIEF DESCRIPTION OF THE DRAWINGS

FIG. 1 illustrates a RF and field gradient pulse sequence for measuring the ¹⁵N R₂(1/τ_{CPMG}) dispersion profile with sensitivity enhancement and compensation of RF heating effects;

FIG. 2 illustrates a two-site and three-site exchange mechanisms for the interaction of N-acetyl-Hir(55-65) with human prothrombin;

FIG. 3 illustrates ¹⁵N relaxation dispersion profiles for the residues Asp55 (▪), Phe56 (x), Glu57 (●), Glu58 (□) and Ile59 (▴) of the free ¹⁵N-labeled N-acetyl-Hir(55-65) peptide at 298 K and 800 MHz;

FIG. 4 illustrates ¹⁵N relaxation dispersion curves for the ¹⁵N-labeled peptide, N-acetyl-Hir*D56*F*E*E*IP₆₀EEYLQ₆₅, in complex with human prothrombin;

FIGS. 5A and 5B illustrate fits of ¹⁵N relaxation dispersion curves for the peptide N-acetyl-Hir(55-65) in complex with human prothrombin to three-site exchange schemes in which eighteen curves were fitted simultaneously to experimental data using the “linear” (FIG. 5A) and the “full” (FIG. 5B) three-site exchange scheme shown in FIG. 2;

FIG. 6 illustrates the temperature dependence of the apparent k_{off} values for residues Phe₅₆ (●), Glu₅₇ (▪), Glu₅₈ (□), and Ile₅₉ (◯) in the complex between the N-acetyl-Hir(55-65) peptide and human prothrombin;

FIG. 7 illustrates ¹⁵N relaxation dispersion curves for the residues Phe₅₆ (♦), Glu₅₇ ( [symbol] ), Glu₅₈ (◯), Ile₅₉ (□), Glu₆₁ (Δ) and Tyr63 (▪) of the free uniformly ¹⁵N-labeled peptide Hir(54-65) at 288 K and 800 MHz;

FIGS. 8A to 8F illustrate ¹⁵N relaxation dispersion curves for selected amide nitrogen atoms of the uniformly ¹⁵N-labeled recombinant Hir(54-65) peptide in complex with human prothrombin at 288 K;

FIGS. 9a to 9D illustrate ¹⁵N relaxation dispersion data at 500 MHz (⋄; ¹⁵N frequency: 50.684 MHz) and 800 MHz (□; ¹⁵N frequency: 81.076 MHz), and fitted profiles for selected amide nitrogen atoms of the ¹⁵N-labeled peptide, N-acetyl-Hir(55-65), in complex with human α-thrombin in 50 mM NaCl and 50 mM sodium phosphate at pH 5.5, and in the presence of 10% D₂O;

FIGS. 10A and 10B illustrate [¹H—¹⁵N]—HSQC spectra of a mixture of six uniformly ¹⁵N-labeled hexa/pentapeptides, GLDPRH_{L}, GVDPRH_{L}, GFNPRH_{L}, GPNPRH_{L}, GFSARH_{L}, GVSPR, where a one-letter code is used to define the amino acid sequence, and H_{L} stands for homoserine lactone, in the absence (FIG. 10A), and presence (FIG. 10B) of the N-acetyl-Hir(55-65) peptide and human thrombin;

FIGS. 11A and 11B illustrate ¹⁵N relaxation dispersion curves for the mixture of the N-acetyl-Hir(55-65) peptide with six uniformly ¹⁵N-labeled pentapeptides, GLDPR, GVDPR, GFNPR, GPNPR, GFSAR, and GVSPR, in complex with human thrombin;

FIGS. 12A to 12H illustrate ¹⁵N relaxation dispersion curves for selected amide nitrogen atoms of the uniformly ¹⁵N-labeled FD22 thrombin-cleaved peptide in complex with human thrombin at a peptide concentration of ˜0.9 mM in 60 mM sodium phosphate buffer, 0.2 mM EDTA, at pH 5.5, and at 288 K;

FIGS. 13A and 13B illustrate titration experiments showing the effect of adding the mCRIB peptide to cCRIB in the presence of Cdc42;

FIGS. 13C to 13F illustrate dispersion curves of the mCRIB peptides in the presence of (red) Cdc42and (black) Cdc42 and cognate cCRIB peptide;

FIG. 14 illustrates the CRIB containing peptides fragments used in Examples 5 and 7;

FIGS. 15A and 15B illustrate [¹H—¹⁵N] HSQC spectrum (FIG. 15A) and human prothrombin-induced ¹⁵N relaxation dispersion curves for the backbone ¹⁵N nuclei of residues Phe₅₆ (◯), Glu₅₇ (▾), and Glu₅₈ (●) (FIG. 15B) of the peptide Hir(54-65) at 288 K;

FIGS. 16A to 16D represent ¹⁵N relaxation dispersion curves for a mixture of mSte20 and mCla4 competing for the same binding site on Cdc42;

FIGS. 17A to 17D illustrate perturbations of Cla4 and Ste20 peptide fragments (¹⁵N-mCla4+Cdc42 [FIG. 17A], ¹⁵N-cCla4+Cdc42 [FIG. 17B], ¹⁵N-mSte20+Cdc42 [FIG. 17C], and ¹⁵N-cSte20+Cdc42 [FIG. 17D] by Cdc42;

FIGS. 18A to 18D represent ¹⁵N relaxation dispersion curves for ¹⁵N-mCla4 free (red) and in complex with Cdc42 (ratio ˜10:1)(black)(FIGS. 18A and 18B) and best fit curves generated from simultaneous fits to data recorded at two magnetic field strengths (800 MHz and 500 MHz) on the mCla4-Cdc42 complex (FIGS. 18C and 18D);

FIGS. 19A to 19D represent ¹⁵N relaxation dispersion curves for ¹⁵N-mSte20 free (red) and in complex with Cdc42 (ratio ˜10:1)(black)(FIGS. 19A and 19B and best fit curves generated from simultaneous fits to data recorded at two magnetic field strengths (800 MHz and 500 MHz) on the mSte20:Cdc42 complex (FIGS. 19C and 19D);

FIG. 20A illustrates the fragmentation of human cathepsin B propeptide producing the amino acid sequences of the wild-type full-length sequence (WT), methionine-introducing mutant (Mutant) and the CNBr-cleaved fragments (F1-F5) of the propeptide wherein S^{H} indicates homoserine lactone; and

FIGS. 20b and 20C illustrate the [¹H—¹⁵N]—HSQC spectrum of the ¹⁵N-labeled recombinant propeptide with the wild-type amino acid sequence (FIG. 20B and of the F1-F5 peptide mixture (FIG. 20C).

### DETAILED DESCRIPTION OF THE PREFERRED EMBODIMENT

The following analysis of ¹⁵N NMR relaxation dispersion reveals that it is possible to determine the dissociation rate constant k_{off} of protein-ligand complexes from the NMR relaxation dispersion curves obtained with two or more external magnetic fields and at one or more protein concentrations. More importantly, it is found that the accuracy of the ligand or protein concentrations is not critical for the data analysis, as only the increases in the protein/ligand ratio, measured by the volumes of the titrated protein solution, need to be included in the data fitting process. In particular, it can be shown that if [L_{free}]>>K_{D} and [L₀]>>[E₀],
kₒₙ′(second)=[(V_{stock}¹+V_{stock}²)/V_{stock}¹]×kₒₙ′(first)
where V_{stock}¹ is the volume of the protein stock solution used in the first addition, V_{stock}² is the volume of the protein stock solution used in the second addition, etc. Any other method of titration allowing determination of the proportionality between the kₒₙ′ values at different protein concentrations is also valid.

Simultaneous fitting of the dispersion curves obtained at multiple protein concentrations makes it possible to identify ligand moieties involving more complex exchange mechanisms (including one free and two or more bound states). The nuclei (or ligand moieties) following a two-state binding-dissociation mechanism behave similarly to each other and display reasonable physical parameters, such as k_{off}, p_{b} (apparent fraction of the bound peptide), R_{2b}(500), R_{2b}(800)(apparent R₂ relaxation rate of the peptide ¹⁵N nuclei in the bound stateat either 500 or 800 MHz), and δω_{bf} (frequency separation of a peptide ¹⁵N signal in the free and bound states: δω_{b-}δω_{f}), allowing a more accurate determination of the dissociation rate constants through combined data fitting.

The data analysis procedure can be easily automated, including input of the relaxation dispersion data, model fitting, identification of the sites with two-state binding behavior and calculation of the k_{off} values at multiple protein titrations. In practical implementations it should be kept in mind that the CPMG NMR relaxation dispersion experiments, including ¹⁵N relaxation as illustrated here as well as NMR relaxation of other nuclei, such as ¹H, ¹⁹F, ¹³C, ³¹P, etc., have a limited sensitivity to the time scales of the ligand dissociating process, i.e. from tens of milliseconds to hundreds of microseconds. As well, quantitative analysis using the proposed titration procedure without precise knowledge of the ligand and protein concentrations requires the large excess of the ligand over the binding protein and that the ligand concentration be much larger than the dissociation constant of the protein complex (see Eqn 3). In some cases, faster ligand off-rates may be quantitated by the NMR T₁ₚ experiments (Mulder, F. A., van Tilborg, P. J., Kaptein, R., and Boelens, R. (1999) J.Biomol.NMR 13, 275-288; and Trott, O. and Palmer, A. G., Ill (2002) J.Magn Reson. 154, 157-160) and multiple quantum relaxations (Wang, C. and Palmer, A. G. (2002) J.Biomol.NMR 24, 263-268).

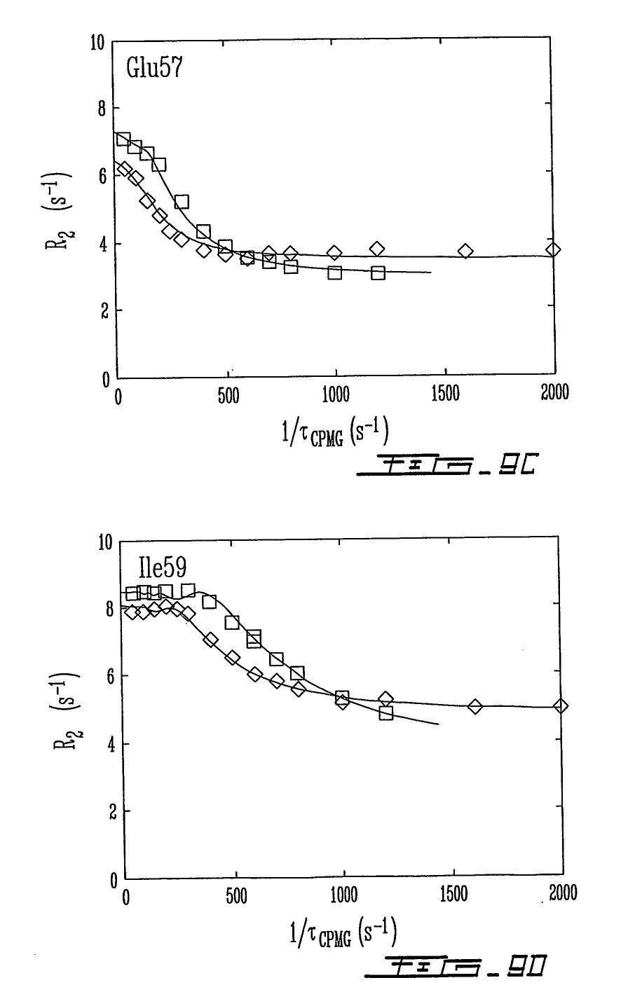

The new NMR techniques according to the present invention have now also been applied to a mixture of peptides either binding to distinct sites or competing for one site on a target protein (FIGS. 11-16).

Materials and Methods

Protein Preparation

Stock solutions of human prothrombin and thrombin were prepared as described previously (Carlisle, T. L., Bock, P. E., and Jackson, C. M. (1990) J.Biol.Chem. 265, 22044-22055; and Fenton, J. W., Fasco, M. J., and Stackrow, A. B. (1977) J.Biol.Chem. 252, 3587-3598; and Ni, F., Ning, Q., Jackson, C. M., and Fenton, J. W. (1993) J.Biol.Chem. 268, 16899-16902). Immediately before use, human thrombin was thawed on ice and concentrated to ˜8 mg/ml using Centricon-10 concentrator (Amicon).

Recombinant Cdc42 (residues 1-178) from Candida Albicans (CaCdc42) was expressed as a His₆-tag protein in Escherichia coli BL21 (DE3) (Novagen) using a pET15b-CaCdc42 expression plasmid engineered with a thrombin cleavage site. Uniform enrichment of CaCdc42 with ¹⁵N and/or ¹³C was achieved by growing the bacteria in minimal medium supplemented with ¹⁵(NH₄)₂SO₄ and/or ¹³C₆-glucose as the sole nitrogen and carbon sources. His₆-CaCdc42 was purified from bacterial lysate by absorption onto a Ni—NTA column (Qiagen) under native conditions (20 mM Tris-HCl, 500 mM NaCl and 2 mM MgCl₂ at pH 8.0, 15 mM immidazole) and eluted with a 30 mM to 300 mM immidazole gradient. Following buffer exchange into 20 mM Tris-HCl, 2 mM MgCl₂ at pH 8.0, the His₆-tag was removed with thrombin and the thrombin inhibitor PPACK added to halt the cleavage reaction. CaCdc42 was obtained after application to a Q-Sepharose™ column and eluted with a 0 to 400 mM NaCl gradient. An activated form of Cdc42 was generated using the non-hydrolysable GTP analogue, β,γ-methyleneguanosine 5′-triphosphate, or GMPPCP (SIGMA). Due to the similar affinity of GDP and GMPPCP for Cdc42, alkaline phosphatase beads were used to degrade GDP as follows. The GDP-loaded form of CaCdc42 was exchanged into a buffer that was 20 mM in Tris, 2 mM MgCl₂, at pH 8.0. Ammonium sulfate was added to a final concentration of 0.2 M. Excess Mg²⁺ was removed by adding 20 mM EDTA. GMPPCP was then added to a 10-fold molar excess of Cdc42 and the mixture added to alkaline phosphatase beads (˜100 units) and shaken gently for 2 hours. The beads were then removed by low speed centrifugation and 25 mM MgCl₂ were added. NMR samples were obtained after exchange into a buffer containing 50 mM phosphate, 2 mM MgCl₂, 50 mM NaCl, pH 6.15 using a PD-10 column.

Peptide Preparation

The peptide N-acetyl-*D₅₅F*E*E*IP₆₀EEYLQ₆₅ (N-acetyl-Hir(55-65)) was enriched selectively with ¹⁵N isotopes at the amides of five residues Asp₅₅, Phe₅₆, Glu₅₇, Glu₅₈ and Ile₅₉ as described previously (Carpenter, K. A. and Ni, F. (1992) J.Magn.Reson. 99, 192-197). Dried powder of the purified peptide was weighed using a Sartorius Supermicro S4™ balance and dissolved at ˜1.5 mM in an aqueous solution (10% D₂O) that was 50 mM in NaCl and 50 mM in sodium phosphate at pH 5.5. Carefully measured volume aliquots of human prothrombin at a stock concentration of 0.3 mM were added to the peptide solution to produce molar ratios of ˜1:45, ˜1:35, and ˜1:30 for the prothrombin and peptide concentrations, respectively.

A small fusion sequence, termed SFC120, was used as the carrier protein to express all the ¹⁵N-labeled peptides used in this application. SFC120 was adopted for peptide production from the N-terminal oligonucleotide binding domain of M. ribonuclease HL which comprises 120 amino acid residues. The cDNA was amplified by standard PCR methods while the restriction enzyme site of Nco I was generated in the 5′-end and the two restriction enzyme sites of EcoR I and BamH I were generated in the 3′-end. The PCR product was double-digested by Nco I and BamH I and ligated into the pET15M vector, which was modified from the pET-15b vector (Novagen) by removing the EcoR I site. The constructed fusion protein expression vector was termed as pTSN-6A.

The DNA fragments encoding the peptides were amplified from a cDNA library by PCR or synthesized as oligonucleotides using the codon preference of E coli. The DNA fragments were digested with EcoR I and BamH I, and subcloned into the pTSN-6A vector. The expression constructs were transformed into the BL21(DE3) expression host and confirmed by DNA sequencing. A single methionine residue was inserted between the fusion protein and the desired peptide sequence to facilitate release of the peptides by CNBr cleavage. A His-tag with six histidines can be placed at the N-terminus of SFC120 to allow purification of the fusion protein by adsorption onto a Ni—NTA agarose column (QIAGEN). In the present case, the His-tagged SFC120 vector was used to express the peptide FD22 (see Examples 4 and 7). Non-His-tagged SFC120 was used to express the rest of the peptides.

Expression of the peptide fragments was achieved by transformation of the appropriate plasmid into E. coli BL21(DE3) competent cells. An overnight culture grown in 2YT containing 100 μg/ml ampicillin (25 ml) was used to inoculate 1 L of M9 minimal media (100 μg/ml ampicillin) supplemented with BME vitamins solution (10 ml/L of 100× stock—SIGMA). ¹⁵N-labeled peptides were expressed using ¹⁵(NH₄)₂SO₄ (2 g/L) as the sole nitrogen source. The cells were grown at 37° C. to a cell density of OD₆₀₀=0.8 and induced by adding IPTG to a final concentration of 1 mM. The cells were incubated for 4-12 hours at 37° C. and collected by centrifugation (8000 g for 20 minutes).

Cell pellets were resuspended in 6 M urea in 20 mM Tris, 100 mM NaCl buffer, pH 8.0 for 4 hours and then sonicated for 45 seconds on ice. The solution was then centrifuged at 7 K rpm for 20 minutes. An equivolume of 100% cold ethanol was added to the supernatant and the solution allowed to stand at 4° C. for two hours. After centrifugation, another equivolume of cold ethanol was added to the supernatant and allowed to stand overnight. The solution was centrifuged at 8,500 rpm and the pellet containing the pure fusion-peptide fragment subjected to SDS-page analysis. If necessary the pellet was further resuspended in 6 M urea and applied to a Sep-Pak™ column (Waters) to remove any impurities. The fusion protein was then lyophilized.

An additional step of purification on a Ni—NTA agarose column for His-tagged fusion peptides was performed as follows. Cell pellets were resuspended in 6 M urea in Tris-HCL buffer at pH 8.0 by gentle shaking for ˜4 hours and briefly sonicated on ice. After centrifugation at 7 K rpm for 20 minutes the supernatant was applied to a Ni—NTA agarose column (QIAGEN) previously equilibrated with the lysis buffer. The column was then washed with ˜20 column volumes of 6 M urea in Tris buffer at pH 6.3 to eliminate non-specific binding to the column. The His-tagged fusion protein was then eluted with 6 M urea in 20 mM Tris buffer at pH 4.5. The solubilized fusion protein was then lyophilized to dryness.

CNBr cleavage was used to release the target peptide from the fusion protein as follows. The fusion protein was dissolved in 70% TFA, CNBr added to a final molar ratio of 100:1 and the solution allowed to stand for ˜24 hours. The samples were then diluted with water (×10) and lyophilized to dryness and purified by RP-HPLC on a C18 column using an acetonitrile-water gradient containing 0.1% TFA. The peptides were lyophilized and there identity was confirmed by electrospray mass spectrometry. Free peptides were prepared for NMR analysis by resuspending the lyophilized peptides into the appropriate NMR buffer solution.

The mixture of the fragments F1, F2, F3, F4, and F5 of human cathepsin B were prepared as follows. The fusion protein was purified by GST affinity chromatography followed by the proteolytic removal of the carrier protein using thrombin as the cleavage enzyme. The intact mutant propeptide was further purified by HPLC and cleaved by CNBr in the solution of 50% formic acid for 24 hours at room temperature. The peptide mixture was desalted either by dialysis or by a Sep-Pak™ reversed-phase C₁₈ column. The peptide mixture was then lyophilized and dissolved in 50 mM sodium acetate-d₃ buffer, pH 5.5-6.0.

Titration of the thrombin-cleaved FD22 peptide was carried out as follows. The sample of FD22 was concentrated by Speed-Vac to 40 μl and 180 μl of a concentrated thrombin solution were added to give a final thrombin:peptide ratio of approximately 1:20 in 60 mM sodium phosphate buffer, 0.2 mM EDTA, 10% D₂O, at pH 5.5. Additional steps of thrombin titrations were carried out by the addition of the human □-thrombin concentrated to ˜8 mg/ml.

NMR Signal Assignments of the ¹⁵N-Labeled Peptides

Proton resonance assignment for the N-acetyl-Hir(55-65) peptide was achieved by using 2D NOESY-[¹H,¹⁵N]—HSQC with an NOE mixing time of 250 ms and 2D TOCSY-[¹H,¹⁵N]—HSQC with a TOCSY mixing time of 56.6 ms (Cavanagh, J., Fairbrother, W. J., Palmer, A. G., and Skelton, N. J. (1995) Protein NMR Spectroscopy: Principles and Practice. Academic Press, San Diego) spectra recorded at 288 K and 500 MHz. Amino acid residues were identified on the basis of the cross-peak patterns from the TOCSY spectrum, and assigned through sequential NOE connectivities. ¹⁵N resonances were assigned using a [¹H—¹⁵N] HSQC (Cavanagh, J., Fairbrother, W. J., Palmer, A. G., and Skelton, N. J. (1995) Protein NMR Spectroscopy: Principles and Practice. Academic Press, San Diego) spectrum acquired at 288 K and 500 MHz.

Proton resonance assignment for the recombinantly expressed ¹⁵N-labeled Hir(54-65) peptide was achieved by using 2D NOESY-[¹H,¹⁵N]—HSQC with an NOE mixing time of 250 ms and 2D TOCSY-[¹H,¹⁵N]—HSQC with a TOCSY mixing time of 60.48 ms spectra recorded at 288 K and 800 MHz. Amino acid residues were identified on the basis of the cross-peak patterns from the TOCSY spectrum, and assigned through sequential NOE connectivities. ¹⁵N resonances were assigned by using a 2D [¹H,¹⁵N]—HSQC-TOCSY spectrum with a TOCSY mixing time of 55.76 ms (Cavanagh, J., Fairbrother, W. J., Palmer, A. G., and Skelton, N. J. (1995) Protein NMR Spectroscopy: Principles and Practice. Academic Press, San Diego) acquired at 288 K and 800 MHz.

Resonances of the thrombin-cleaved ¹⁵N-labeled FD22 peptide were assigned by use of two homonuclear experiments, NOESY with an NOE mixing time of 250 ms and TOCSY with a TOCSY mixing time of 58.32 ms, were recorded at 288 K and 500 MHz. In both experiments, ¹⁵N decoupling was applied during the t1 and t2 periods, and the water resonance was flipped-back to the +Z axis prior to data acquisition (Lippens, G., Dhalluin, C., and Wieruszeski, J.-M. (1995) J.Biomol.NMR 5, 327-331; and Fulton, D. B., Hrabal, R., and Ni, F. (1996) J.Biomol.NMR 8, 213-218; and Fulton, D. B. and Ni, F. (1997) J.Magn Reson. 129, 93-97). Amino acid residues were identified on the basis of the cross-peak patterns from the TOCSY spectrum, and assigned through sequential NOE connectivities. ¹⁵N resonances were assigned by using 2D [¹H,¹⁵N]—HSQC-TOCSY with a TOCSY mixing time of 55.44 ms acquired at 288 K and 800 MHz.

Assignments for the ¹⁵N labeled CRIB peptides were obtained in a sequential manner from homonuclear ¹H—¹H 2D and 3D ¹⁵N-edited TOCSY and NOESY spectra.

Measurements of the ¹⁵N NMR Relaxation Dispersion Profiles

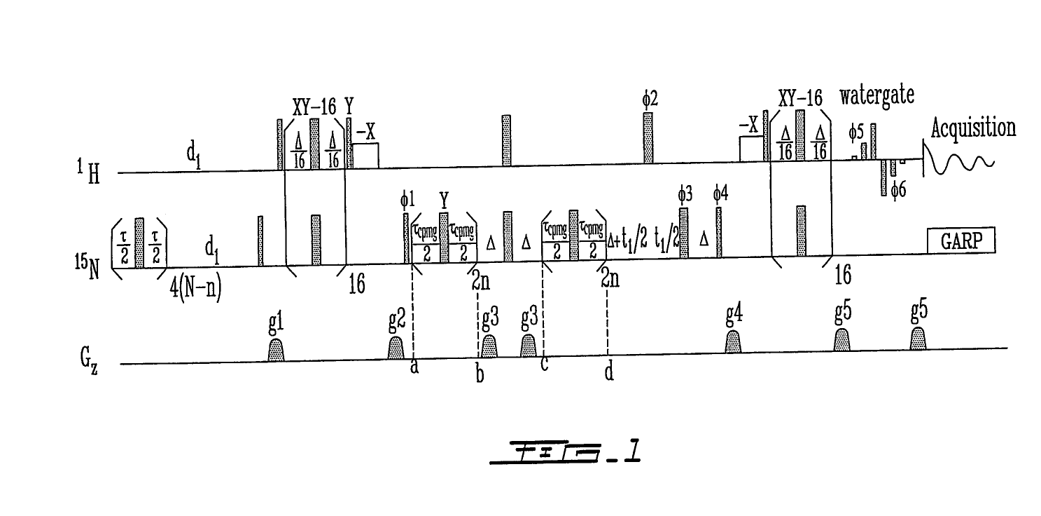

The sample temperatures were calibrated using methanol (Cavanagh, J., Fairbrother, W. J., Palmer, A. G., and Skelton, N. J. (1995) Protein NMR Spectroscopy: Principles and Practice. Academic Press, San Diego). The core of the NMR relaxation dispersion measurements is the relaxation-compensated CPMG pulse scheme (FIG. 1) (Millet, O., Loria, J. P., Kroenke, C. D., Pons, M., and Palmer, A. G. (2000) J.Am.Chem.Soc. 122, 2867-2877; and Loria, J. P., Rance, M., and Palmer, A. G. (1999) J.Am.Chem.Soc. 121, 2331-2332). A train of ¹⁵N 180° pulses with a separation of 1 ms is applied at 100 ppm off the center of the HSQC spectrum at the beginning of the recycle delay d₁ such that the total number of ¹⁵N 180° pulses, 4N, is kept the same for all experiments with different CPMG pulse repetition rates. An XY-16 180° pulse train (Gullion, T., Baker, D. B., and Conradi, M. S. (1990) J.Magn.Reson. 89, 479-484) is also applied to both the ¹H and ¹⁵N nuclei during the two INEPT periods, which limits the decay of exchange-broadened ligand resonances. Further enhancement in sensitivity was achieved by using the 3919 WATERGATE sequence for water suppression (Piotto, M., Saudek, V., and Sklenar, V. (1992) J.Biomol.NMR 2, 661-665). The 90° and 180° RF pulses are represented by narrow and wide bars, respectively, applied along the +X axis unless specified otherwise. The open rectangles are water-selective soft pulses with duration of 2 ms. During data acquisition, ¹⁵N decoupling is achieved using a GARP sequence (Shaka, A. J., Barker, P. B., and Freeman, R. (1985) J.Magn.Reson. 64, 547-552) with an RF field of 1.0 kHz (at 500 MHz) or 1.2 kHz (at 800 MHz). The delays are: Δ=2.7 ms, τ=1 ms, d₂=d₁-4(N-n)τ and τ_{CPMG}=(T/4n−pw180_{N}), where T is the total duration of the ¹⁵N CPMG pulse train and pw¹⁸⁰_{N} is the width of the ¹⁵N 180° pulse. Sinebell-shaped gradient pulses with a duration of 1 ms are used with gradient strengths of g₁=5 G/cm, g₂=6 G/cm, g₃=1.2 G/cm, g₄=6 G/cm, and g₅=10 G/cm. The phases of some RF pulses are the same as those reported previously with φ1=+X−X, φ2=4(+X) 4(−X) and φ3=2(+X) 2(+Y) 2(−X) 2(−Y) (Millet, O., Loria, J. P., Kroenke, C. D., Pons, M., and Palmer, A. G. (2000) J.Am. Chem. Soc. 122, 2867-2877), except that the phases for the last ¹⁵N 90° pulse, the WATERGATE sequence and the RF receiver are cycled as φ4=4(+X) 4(−X), φ5=8(+X) 8(+Y), φ6=8(−X) 8(−Y) and receiver=(+X−X−X+X) 2(−X+X+X−X) (+X−X−X+X). Phase-sensitive ¹H—¹⁵N HSQC spectra were obtained by incrementing φ4, according to the States-TPPI scheme (Marion, D., Ikura, M., Tschudin, R., and Bax, A. (1989) J.Magn.Reson. 85, 393-). The relaxation dispersion profile is derived from the spectral peak intensities at different effective B₁ fields (Blackledge, M. J., Bruschweiler, R., Griesinger, C., Schmidt, J. M., Xu, P., and Ernst, R. R. (1993) Biochemistry 32, 10960-10974), expressed by the equation R₂(1/τ_{CPMG})=−ln[l(1/τ_{CPMG})/l(0)]/T (Mulder, F. A. A., Skrynnikov, N. R., Hon, B., Dahlquist, F. W., and Kay, L. E. (2001) J.Am.Chem.Soc. 123, 967-975), where l(1/τ_{CPMG}) is the intensity of an HSQC peak with varying n, hence the τ_{CPMG} delay, and l(0) is the intensity of the same HSQC peak in the absence of the ¹⁵N CPMG pulse trains (i.e. n=0 in periods a-b and c-d). NMR data were collected at ¹⁵N frequencies of 50.684 and 81.076 MHz using Bruker Avance/DRX 500 and 800 MHz NMR spectrometers. The total length of the ¹⁵N CPMG pulse train, T (FIG. 1), is kept at a constant value of 40 ms and the total number (4N) of the ¹⁵N CPMG 1800 pulses was set to 100.

Fitting of the NMR Relaxation Dispersion Profiles and Derivation of the Dissociation Rate Constants k_{off}

Under the experimental conditions, the NMR magnetization M in the rotating frame was assumed to evolve between the 180° refocusing pulses according to the following equations:

$$\begin{matrix}ⅆ M / ⅆ t = ( R + {R}_{EX} ) M ; & ( 1 ) \\ where R = ( \begin{matrix}- {R}_{2 b} - i {δω}_{b} & 0 \\ 0 & - {R}_{2 f} - i {δω}_{f}\end{matrix} ) ; & ( 1 a ) \\ {R}_{EX} = ( \begin{matrix}- {k}_{off} & {k}_{on}^{′} \\ {k}_{off} & - {k}_{on}^{′}\end{matrix} ) ; & ( 1 b )\end{matrix}$$

for a two-state exchange model, and

$$\begin{matrix}R = ( \begin{matrix}- {R}_{2 b} - i {δω}_{b 2} & 0 & 0 \\ 0 & - {R}_{2 b} - i {δω}_{b 1} & 0 \\ 0 & 0 & - {R}_{2 f} - i {δω}_{f}\end{matrix} ) ; & ( 1 c ) \\ {R}_{EX} = ( \begin{matrix}- {k}_{1} - {k}_{off}^{2} & {k}_{2} & {k}_{on}^{′2} \\ {k}_{1} & - {k}_{2} - {k}_{off}^{1} & {k}_{on}^{′ 1} \\ {k}_{off}^{2} & {k}_{off}^{1} & - {k}_{on}^{′ 1} - {k}_{on}^{′2}\end{matrix} ) ; & ( 1 d )\end{matrix}$$

for a three-state exchange model (Jen, J. (1978) J.Magn Reson. 30, 111-128).

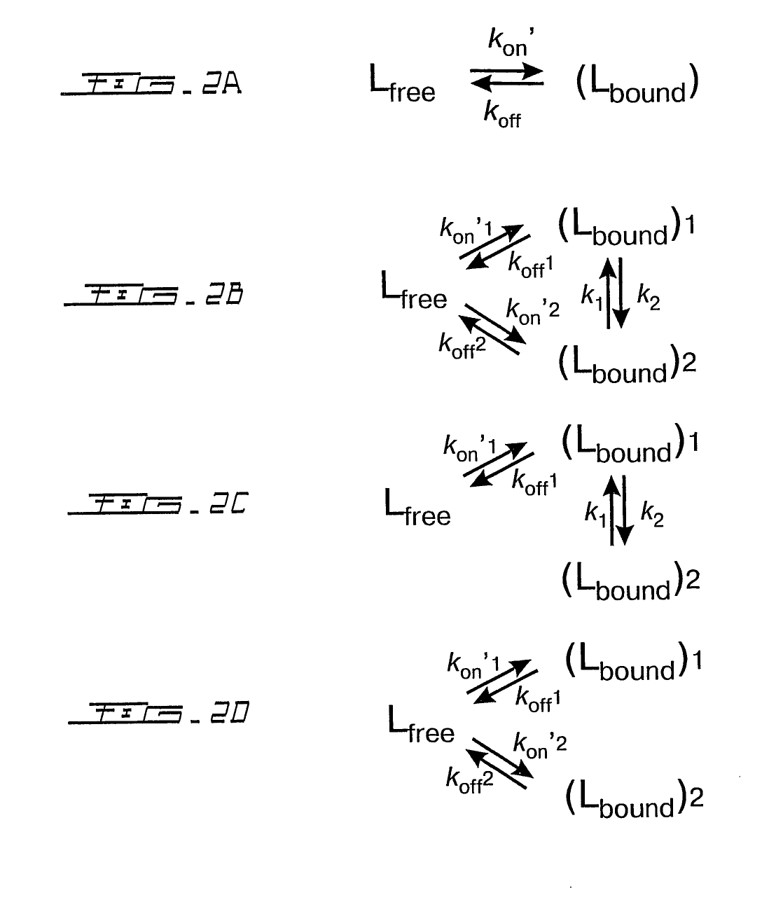

The notation for the exchange rate constants is shown in FIG. 2. In FIG. 2, addition of an extra bound state to the two-state exchange model (pathway A) produces a “full” three-site exchange model (pathway B) with two additional exchange pathways. “Full” three-site exchange scheme turns into “linear” (pathway C) and “forked” (pathway D), if the corresponding absent exchange pathways are too slow to be sensed by NMR. In the cases of “linear” (pathway C) or “forked” (pathway D) three-state exchange schemes, rate constants between non-exchanging species (kₒₙ^{—2}, k_{off}² or k₁, k₂, respectively) were set to zero. R_{2b} and R_{2f} are the intrinsic transverse relaxation rates for the peptide ¹⁵N nuclei in the bound and free states, respectively. The transverse relaxation rates for the bound peptide in the two possible complexes were assumed to be equal. Variables δω_{b}, δω_{b2} and δω_{b1} are the resonance frequency offsets (ω-ω₀) for the corresponding bound states, δω_{f} is the resonance frequency offset for the free peptide, and ω₀ is the angular frequency of the CPMG RF pulse train.

The two-state exchange model can be fit to the experimental relaxation dispersion curves by use of a single exponential approximation, which is valid for all exchange conditions when the concentration of the free ligand is in large excess over that of the bound states (Carver, J. P. and Richards, R. E. (1972) J.Magn.Reson. 6, 89-105; and Jen, J. (1978) J.Magn Reson. 30, 111-128; and Davis, D. G., Perlman, M. E., and London, R. E. (1994) J.Magn Reson.B 104, 266-275; and Ni, F. (1994) Progress in NMR spectroscopy 26, 517-606). In this case the following equations relate the NMR relaxation dispersion profiles to the underlying physical parameters (Carver, J. P. and Richards, R. E. (1972) J.Magn.Reson. 6, 89-105; and Jen, J. (1978) J.Magn Reson. 30, 111-128; and Davis, D. G., Perlman, M. E., and London, R. E. (1994) J.Magn Reson.B 104, 266-275),
R₂^{eff}−ln[I(1/τ_{CPMG})/I(0)/T=(R_{2f}+R_{2b}k_{off}kₒₙ′)/2−(1/τ_{CPMG})ln λ+ (2)
where
lnλ+½cosh⁻¹[D₊cos2ξ−D−cos2η]; (2a)
D±=½[±1+(ψ+2δω_{bf}²)/(ψ²ξ²)^{1/2}]; (2b)
ξ=(τ_{CPMG}/√8)[+ψ+(ψ²+ξ²)^{1/2}]^{1/2}; (2c)
η=(τ_{CPMG}/√8)[−ψ+(ψ²+ξ²)^{1/2}]^{1/2}; (2d)
ψ=(R_{2f}−R_{2b}+kₒₙ′−k_{off}²−δω_{bf}²+4kₒₙ+k_{off} (2e)
ξ=δω_{bf}(R_{2f}−R_{2b}+kₒₙ′−k_{off}) (2f)
δω_{bf}=δω_{b}−δω_{f}. (2g)

For three-site exchange situation, the magnetization evolution can be obtained by numeric integration of the equations (1) and fit to the experimental NMR relaxation data (Jen, J. (1978) J.Magn Reson. 30, 111-128; and Tollinger, M., Skrynnikov, N. R., Mulder, F. A., Forman-Kay, J. D., and Kay, L. E. (2001) J.Am;Chem.Soc. 123, 11341-11352). For the specific application herein, values for k_{off}, kₒₙ′, k_{off}¹, k_{off}², kₒₙ⁻¹, k₁, k₂, R_{2b}(500 MHz), R_{2b}(800 MHz), δω_{b}, δω_{b1} and δω_{b2} (FIG. 2) were fitted as independent variables. Values kₒₙ⁻², p_{b}, p_{b1}, p_{b2}, and p_{f}=(1−p_{b1}−p_{b2}), with p standing for the population of the corresponding species, were derived using the condition of microscopic reversibility kᵢⱼpᵢ=kⱼᵢpⱼ, where kᵢⱼ denotes the exchange rate constant for the transformation of species i into species j. R_{2f}, or the intrinsic R₂ relaxation rate of the peptide ¹⁵N nuclei in the free state, and δω_{f} are determined experimentally for a sample of the peptide alone under the same experimental conditions.

Error Evaluation

The errors of fitting the kinetic and NMR relaxation parameters using Equations 1 and 1a to 1d were estimated through Monte-Carlo sampling as follows. Random deviations of the measured peak integral intensities were generated for each of the 200 Monte-Carlo samples. The absolute value of the deviation was set to 1% of the intensity corresponding to zero relaxation delay, which was a justifiable uncertainty based on a few independent experiments. Error analysis for curve fitting using Equations 2 and 2a to 2g was carried out in a slightly different manner. The standard deviations of the fitted parameters were estimated through (bootstrap) Monte Carlo simulations (Press, W. H., Teukolsky, S. A., Vetterling, W. T., and Flannery, B. P. (1992) Numerical Recipes in Fortran. Cambridge University Press) using a maximum deviation of ±10% for all experimental R₂ values.

Quantitative Analysis of Ligand NMR Relaxation Dispersion Profiles of Transient Protein-Ligand Complexes

¹⁵N NMR Relaxation Dispersion Profiles of an Anti-Thrombin Peptide Interacting With Human Prothrombin.

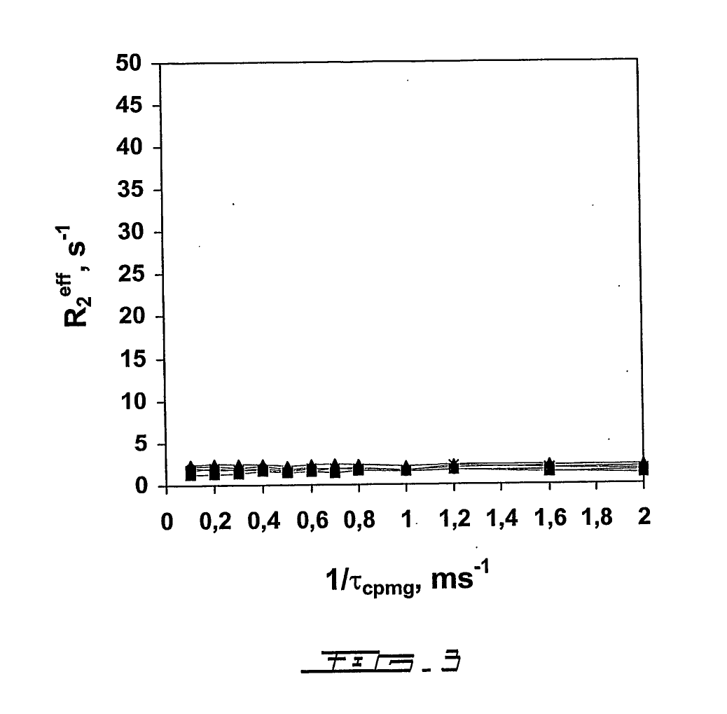

The anti-thrombin peptide N-acetyl-*Asp-*Phe-*Glu-*Glu-*Ile-Pro-Glu-Glu-Tyr-Leu-Gln (SEQ ID NO:15)(to be referred to as N-acetyl-Hir(55-65) in the subsequent descriptions), contains five ¹⁵N-labeled residues Asp₅₅, Phe₅₆, Glu₅₇, Glu₅₈ and Ile₅₉ at their backbone amide nitrogens. The peptide free in solution displays slowly relaxing (sharp) NMR signals with no detectable response (FIG. 3) of the ¹⁵N transverse relaxation rate to the changes in CPMG pulse rate using the NMR pulse sequence in FIG. 1. In FIG. 3, the peptide was ˜1.5 mM in an aqueous solution (10% D₂O) that was 50 mM in NaCl and 50 mM in sodium phosphate at pH 5.5. Upon addition of a small amount of prothrombin (less than 1:50 protein/ligand molar ratio), four of the five {¹H—¹⁵N}—HSQC peaks were found to significantly shift and broaden. For all exchange regimes, excess ligand over a large protein-ligand complex guarantees that the NMR spectrum is dominated by the slowly decaying signals resulting from the free ligand (Ni, F. (1994) Progress in NMR spectroscopy 26, 517-606). The ¹⁵N transverse relaxation dispersion data (FIG. 4) were collected using the implementation illustrated in (FIG. 1) of the constant-time ¹⁵N CPMG experiment (Mulder, F. A. A., Skrynnikov, N. R., Hon, B., Dahlquist, F. W., and Kay, L. E. (2001) J.Am.Chem.Soc. 123, 967-975) along with additional CPMG elements for sensitivity enhancement of exchange-broadened signals (Mulder, F. A. A., Spronk, C. A. E. M., Slijper, M., Kaptein, R., and Boelens, R. (1996) J.Biomol.NMR 8, 223-228) and for the compensation of heating effects (Wang, A. C. and Bax, A. (1993) J.Biomol.NMR. 3, 715-720) caused by the CPMG pulses (Mulder, F. A., van Tilborg, P. J., Kaptein, R., and Boelens, R. (1999) J.Biomol.NMR 13, 275-288). The use of compensating ¹⁵N CPMG pulses was found to be critical for running the relaxation dispersion experiments on a very high field NMR spectrometer (such as at 800 MHz) that may be very sensitive to small temperature fluctuations.

FIG. 4 shows the ¹⁵N relaxation dispersion of the backbone amide nitrogen atoms of residues Phe₅₆, Glu₅₇, Glu₅₈ and Ile₅₉ of N-acetyl-Hir(55-65) in the presence of human prothrombin. The amide nitrogen of Asp₅₅ had a rather sharp ¹⁵N NMR signal and showed very little relaxation dispersion, in agreement with the very little binding-induced line broadening of its amide proton resonance (Ni, F., Ning, Q., Jackson, C. M., and Fenton, J. W. (1993) J.Biol.Chem. 268, 16899-16902), and therefore was not included in further analysis. Three prothrombin/peptide molar ratios were used, namely ˜1:45, ˜1:35, and ˜1:30. The accuracy of the absolute peptide or prothrombin concentrations was not critical for the data analysis, as only the increases in the prothrombin/peptide ratio, measured by the volume of the added prothrombin stock solution, need to be included in the data fitting process (vide infra). The approximate prothrombin concentrations were used only to discriminate physically reasonable from unreasonable fits. In the course of the titration the volumes of the added prothrombin solution after the second and third additions were 1.29 and 1.5 times that of the first addition, respectively. The relaxation dispersion data were fitted using numerical calculation of the magnetization evolution (Equations 1 and 1a to 1d) or the single-exponential approximation (Equations 2 and 2a to 2g) during the (τ_{CPMG}/2-180°-τ_{CPMG}-180°-τ_{CPMG}/2) element of CPMG sequence (Jen, J. (1978) J.Magn Reson. 30, 111-128). In FIG. 4, the peptide was ˜1.5 mM in an aqueous solution (10% D₂O) that was 50 mM in NaCl and 50 mM in sodium phosphate at pH 5.5, and at 298 K; The dispersion curves were recorded at three prothrombin:peptide ratios, 1:45 (◯, ●), 1:35 (□, ▪), and 1:30 (⋄, ♦), and at two ¹⁵N frequencies, 50.684 MHz (◯,□, and ⋄) and 81.076 MHz (●,▪, and ♦). The ¹⁵N dispersion curves were fitted to a two-site exchange scheme separately for each residue, but simultaneously for all three prothrombin:peptide ratios. The fitted curves are shown as solid line at 1:45, dotted line at 1:35, and dash-dot line at 1:30 prothrombin:peptide ratios.

Data Analysis Using a Two-Site Exchange Model

A two-site exchange model was fitted independently for every ¹H/¹⁵N cross-peak either at each prothrombin concentration or by combining the data for all three prothrombin concentrations. In this scheme, p_{b} is the bound population of the peptide, δω_{bf} is the ¹⁵N resonance peak separation (δω_{b}−δδω_{f}) between the bound and the free states, R_{2f} is the transverse relaxation rate for the free peptide, R_{2b} is the transverse relaxation rate for the bound peptide, k_{off} is the dissociation rate constant, kₒₙ′=kₒₙ×[E_{f}] is a pseudo-first order binding rate constant, where kₒₙ is the association rate constant, and [E_{f}] is the concentration of the free prothrombin. In turn, the concentration of the free prothrombin [E_{f}] is defined by the concentration of the free peptide [L_{free}], the total concentration of added prothrombin [E₀], and an equilibrium dissociation constant K_{D} (which in the case of a two-state exchange is equal to k_{off}/kₒₙ) as
[E_{f}]=[E₀]K_{D}(K_{D}+[L_{free}]), (3)
which in the case of [L_{free}]>>K_{D}, and [L₀]>>[E₀], is proportional to the [E₀]/[L₀] ratio. A least squares fitting procedure was used to extract the values of R_{2b} at the two magnetic fields, k_{off}, p_{b}, and δω_{bf}. The values of R_{2f}, the transverse relaxation rate for the ¹⁵N-labeled sites of the free peptide, were derived from the CPMG relaxation dispersion profile of the free peptide (FIG. 3).

The results of the fits are presented in Table 1. It is seen that the behavior of residues Phe₅₆ and Ile₅₉ is consistent with a two-site exchange model. Both residues display highly similar k_{off} and R_{2b} values independent of the peptide/prothrombin ratio, and p_{b} is growing roughly proportionally to the added prothrombin. Glu₅₈, on the other hand, has a noticeably different behavior: the calculated k_{off} value decreases with the addition of prothrombin, while R_{2b} increases, and p_{b} is not changing. Glu₅₇ displays some growth of k_{off} and only slight increase in p_{b} upon the incremental growth of the prothrombin concentration. In addition, R_{2b} values for Glu₅₇ at both fields appear to be unusually high, almost doubling the values calculated for other ¹⁵N sites. Regardless, fitting results with residues Phe₅₆ and Ile₅₉ indicate that ¹⁵N-relaxation dispersion data at two magnetic fields and at a single prothrombin concentration can uniquely determine the five unknown parameters, R_{2b}(500), R_{2b}(800), k_{off}, p_{b} and δω_{bf} describing two-state binding.

TABLE 1
Kinetic and ¹⁵N Relaxation Dispersion Parameters of the N-acetyl-Hir(55-65) Peptide
in Complex with Human Prothrombin for a Two-Site Exchange
Scheme at 298 K¹

| Residue | k_{off}, s⁻¹ | δω_{bf}, ppm | p_{b}, % | R_{2b} [R_{2b}(800)], s⁻¹ | | R_{2f} [R_{2f}(800)], s⁻¹ |
|---|---|---|---|---|---|---|
| Phe56/1 | 2030 ± 110 | 3.7 ± 0.2 | 2.01 ± 0.14 | 161 ± 8 | [319 ± 22] | 1.38 [0.95] |
| Phe56/* | 2050 ± 146 | 3.6 ± 0.2 | 2.0 ± 0.2 | 155 ± 11 | [308 ± 30] | |
| Phe56/2 | 2015 ± 110 | 3.9 ± 0.2 | 2.40 ± 0.21 | 199 ± 10 | [304 ± 21] | |
| Phe56/3 | 2150 ± 105 | 4.2 ± 0.2 | 2.53 ± 0.15 | 190 ± 7 | [304 ± 18] | |
| Phe56 | 2080 ± 60 | 4.0 ± 0.1 | 1.80 ± 0.06 | 188 ± 5 | [308 ± 11] | |
| Glu57/1 | 1540 ± 180 | −2.6 ± 0.3 | 1.74 ± 0.20 | 310 ± 35 | [460 ± 60] | 1.60 [1.44] |
| Glu57/* | 1570 ± 209 | −2.6 ± 0.4 | 1.7 ± 0.3 | 310 ± 46 | [454 ± 78] | |
| Glu57/2 | 1820 ± 190 | −3.4 ± 0.3 | 1.74 ± 0.16 | 410 ± 40 | [514 ± 54] | |
| Glu57/3 | 2030 ± 200 | −3.5 ± 0.4 | 1.92 ± 0.21 | 400 ± 40 | [513 ± 58] | |
| Glu57 | 1850 ± 110 | −3.2 ± 0.2 | 1.36 ± 0.08 | 390 ± 20 | [510 ± 35] | |
| Glu58/1 | 2260 ± 100 | 4.2 ± 0.2 | 3.42 ± 0.20 | 136 ± 6 | [255 ± 17] | 2.14 [2.53] |
| Glu58/* | 2437 ± 153 | 4.3 ± 0.3 | 3.2 ± 0.3 | 132 ± 9 | [245 ± 24] | |
| Glu58/2 | 1790 ± 80 | 5.4 ± 0.2 | 3.09 ± 0.12 | 233 ± 7 | [339 ± 14] | |
| Glu58/3 | 1680 ± 80 | 5.3 ± 0.2 | 3.34 ± 0.14 | 259 ± 7 | [367 ± 14] | |
| Ile59/1 | 2060 ± 90 | −4.4 ± 0.2 | 3.04 ± 0.15 | 209 ± 7 | [361 ± 18] | 0.71 [1.03] |
| Ile59/* | 2165 ± 153 | −4.5 ± 0.3 | 3.0 ± 0.2 | 206 ± 11 | [351 ± 27] | |
| Ile59/2 | 2020 ± 80 | −4.9 ± 0.2 | 3.55 ± 0.15 | 260 ± 8 | [340 ± 15] | |
| Ile59/3 | 2260 ± 100 | −4.5 ± 0.2 | 4.41 ± 0.28 | 193 ± 8 | [312 ± 18] | |
| Ile59 | 2160 ± 60 | −4.6 ± 0.1 | 2.90 ± 0.10 | 221 ± 5 | [330 ± 10] | |
| 56/57/59² | 2470 ± 40 | | 2.40 ± 0.05 | 193 ± 4 | [245 ± 6] | |
| ¹The fitted parameters k_{off}, δω_{bf}, p_{b} and R_{2b} represent, respectively, the dissociation rate constant of the protein-peptide complex, the frequency separation for a peptide¹⁵N signal in the free and bound states (δω_{b}-δω_{f}), the apparent fraction of the bound peptide, and the apparent R₂ relaxation rate | | | | | | |
| # of the peptide ¹⁵N nuclei in the bound state. Signs of the δω_{bf} values are derived from the signs of the prothrombin-induced chemical shifts for the | | | | | | |
| # corresponding [¹H-¹⁵N]-HSQC cross-peaks. The listed values R_{2b} are those at 500 MHz (with an ¹⁵N frequency of 50.684 MHz) and the values in the square brackets are those at 800 MHz (¹⁵N frequency: 81.076 MHz). The values of R_{2f}, or the intrinsic R₂ relaxation rate of the peptide ¹⁵N nuclei in the free state, were determined | | | | | | |
| # experimentally for a sample of the peptide in the absence of prothrombin under the same experimental conditions, and were found | | | | | | |
| # independent of the CPMG pulse rate. Residues were fitted independently except for the last row designated as 56/57/59. For every amino acid residue, rows denoted /1, /2, and /3 correspond to data sets at prothrombin:ligand ratios of 1:45, 1:35, and 1:30, respectively. Rows denoted /* show the results of the fitting of the data in rows denoted /1 (˜1:45 prothrombin/peptide ratio) with | | | | | | |
| # a single-exponential approximation (Carver, J. P. and Richards, R. E. (1972) J.Magn.Reson. 6, 89-105; and Jen, J. (1978) J.Magn Reson. 30, 111-128; and Davis, D. G., | | | | | | |
| # Perlman, M. E., and London, R. E. (1994) J.Magn Reson.B 104, 266-275). The last row for every residue, except for Glu₅₈ which strongly deviates from the concentration dependence implied in a two-site model, lists parameters obtained by a simultaneous fit to dispersion data at all three concentrations, with the value in p_{b} column corresponding to the prothrombin:ligand | | | | | | |
| # ratio of 1:45. The row 56/57/59 displays parameters for the simultaneous fit to the relaxation dispersion of three residues Phe₅₆, Glu₅₇ and Ile₅₉ at three prothrombin concentrations. | | | | | | |

²Fitted values of δω_{bf} were 3.1 ± 0.1, (−1.2) ± 1.8, and (−5.5) ± 0.1 ppm, for residues Phe₅₆, Glu₅₇ and Ile₅₉, respectively.

A simultaneous fit of the dispersion curves was then carried out at all three prothrombin concentrations, using the fact, that kₒₙ′ is proportional to [E_{f}], hence to [E₀] (Equation 3). Three matrices R_{EX}(1:45), R_{EX}(1:35), and R_{EX}(1:30) for each prothrombin:peptide ratio were used to fit simultaneously relaxation dispersion curves from three prothrombin concentrations. Matrices R_{EX}(1:35) and R_{EX}(1:30) are produced from, R_{EX}(1:45) by substituting exchange rates kₒₙ′ (or kₒₙ⁻¹ and kₒₙ⁻² in the models with two dissociation routes) with 1.29×kₒₙ′ (or 1.29×kₒₙ⁻¹ and 1.29×kₒₙ⁻²) and 1.5×kₒₙ′ (or 1.5×kₒₙ′ (or 1.5×kₒₙ⁻¹ and 1.5×kₒₙ⁻²), respectively. The ratios 1.29 and 1.5 for R_{EX}(1:35) and R_{EX}(1:30) reflect the increase of prothrombin/ligand ratio in the relationship kₒₙ′˜K_{D}[E₀]/[L₀] under the assumptions of [L_{free}]>>K_{D} and [L₀]>>[E₀] (see equations (3) and (4)), and are obtained using the volumes of the added prothrombin solution in the titration. The populations of different species with increased prothrombin concentrations were recalculated in accordance with the modified kₒₙ′ (or kₒₙ⁻¹ and kₒₙ⁻²) values. The fit was performed independently for each ¹H/¹⁵N cross-peak (FIG. 4 and Table 1). As expected, the fitted dispersion curves for Phe₅₆ and Ile₅₉ were in a good agreement with the two-site exchange model. It was also possible to fit reasonably well the dispersion curves of Glu₅₇ using the same model. Again, for Glu₅₇ fitted R_{2b} values were higher, while p_{b} was smaller than expected (Table 1). It is important to emphasize that the apparent transverse relaxation rate was very sensitive to the increase of [E_{f}] for every pair of the dispersion curves with the addition of prothrombin. It was therefore easy to verify the linearity of [E_{f}] growth with protein addition simply by including it as a fitted parameter. For Phe₅₆, Glu₅₇ and Ile₅₉ ¹⁵N sites, fitted [E_{f}]₁/[E_{f}]₂ values for every pair of prothrombin concentrations was in excellent agreement with the relative volumes of prothrombin solution added, or the ([E₀]/[L₀])₁/([E₀]/[L₀])₂ ratio. This observation is consistent with the previous reports demonstrating that K_{D} for the binding of N-acetyl-Hir(55-65) with prothrombin is of the order of ˜100-300 μM (Ni, F., Ning, Q., Jackson, C. M., and Fenton, J. W. (1993) J.Biol.Chem. 268, 16899-16902; and Anderson, P. J., Nesset, A., Dharmawardana, K. R., and Bock, P. E. (2000) J.Biol.Chem. 275, 16428-16434), and therefore is significantly less than the peptide concentration (˜1.5 mM).

Simultaneous fitting of eighteen dispersion curves at the three concentrations of prothrombin for all three residues Phe₅₆, Glu₅₇ and Ile₅₉ was also performed and is listed in the last row of Table 1. For this particular fitting, equal R_{2b} values were assumed for each residue in the bound state to simplify the calculations. Whereas parameters for Phe₅₆ and Ile₅₉ remained similar to those obtained independently for every residue, the fit forced poor convergence of the parameter δω_{bf} (1.2±1.8 ppm) for Glu₅₇.

It was not possible to obtain a reasonable fit for residue Glu₅₈ using a two-site exchange model at the three prothrombin concentrations (FIG. 4). The apparent transverse relaxation rate of Glu₅₈ does not grow rapidly enough with the addition of prothrombin to be consistent with the two-site exchange model, thus indicating that Glu₅₈ may undergo more complex exchange processes.

Data Analysis Using a Three-Site Exchange Model

The relaxation behaviors of residues Glu₅₇ and particularly Glu₅₈ suggest the presence of other conformations for these residues in the bound state. Although the relaxation dispersion of Glu₅₇ is possible to be fitted by a two-site exchange model at all three prothrombin concentrations, the abnormally high values of R_{2b} as well as comparatively low values of p_{b}, might be artifacts originating from the presence of additional exchange pathways. In fact, even residues Phe₅₆ and Ile₅₉ display R_{2b} values somewhat higher than expected, if It is assumed that in the bound state it is the motions of the complex as a whole that define the transverse relaxation of the ligand. Theoretical values of R₂ for a protein with the molecular mass of prothrombin (72 kDa) and order parameter S=0.8 and 1.0 were estimated to be respectively 95/122 and 119/153 s⁻1 at 500/800 MHz (Luginbuhl, P. and Wuthrich, K. (2002) Progress in NMR spectroscopy 40, 199-247).

In the most general case including additional bound state adds two kinetic pathways to the model (FIG. 2, pathway B). One pathway is the kinetic exchange on the surface of the protein, that can be a consequence of both protein and peptide conformational conversions. Another pathway is an alternative association-dissociation route of the distinct bound species, formally producing an additional pair of k_{off} and kₒₙ rate constants. If one of the two pathways is too slow to influence the relaxation, the system obeys either “linear” three-state behavior (FIG. 2, pathway C), or “forked” three-state behavior (FIG. 2, pathway D). In the present evaluation, it was assumed that the transverse relaxation rate R_{2b} for both bound states are equal. This way, there are a total of nine independent parameters describing the general three-site exchange, while the total number of the degrees of freedom is reduced by one for the “linear” or “forked” three-site exchange mechanisms (FIG. 2 and Equation 1). The increment of the prothrombin concentration during the titration does not increase the total degrees of freedom as long as the volume ratios of the titrated prothrombin are known accurately. In principle, therefore, the nine kinetic and NMR relaxation parameters may be obtained from fitting of only two dispersion data sets at two different prothrombin concentrations. Equation (3) and the linear proportionality of [E_{f}] to (E₀]/[L₀], given [L_{free}]>>K_{D} and [L₀]>>[E₀], remains valid if an apparent K_{D} value is defined as [E_{f}][L_{free}]/[{EL}_{complex}]. Here {EL}_{complex} represents an ensemble of 1:1 protein-ligand complexes, but K_{D} is not equal to k_{off}/kₒₙ for the three-site or more complex exchange situations.

In practice, it was found that the three-state system may be somewhat underdefined, and depending on the starting conditions, the calculation converged to a few clusters of fitted parameters. The clusters were filtered on the basis of physical feasibility, and parameter sets containing negative rate constants or relaxation times, as well as p_{b}=p_{b1}+p_{b2} exceeding or comparable with p_{free}=(1−p_{b}), were not considered. To fit the experimental dispersion curves of Glu₅₇ to a “linear” three-site model (FIG. 2, pathway C), it was necessary to use simultaneously the six dispersion curves for the three concentrations of prothrombin and at two different magnetic fields to obtain convergent results. Depending on the starting conditions for iterative fitting, the fitted parameters in the calculations converged either to the two-site exchange mechanism (k₁≈k₂≈0), or to the actual linear three-site exchange mechanism (k₁=50±30 s⁻¹, k₂=150±70 s⁻¹). The resulting three-site exchange scheme was, in fact, not so much different from the two-site exchange scheme, since the population of the dissociation-competent bound state, p_{b1}, was found to be 1.3±0.1%, close to that found for the two-site mechanism (Table 1). Part of the bound peptide was redistributed into the (L_{bound})₂ state (p_{b2}=4.0±1.8%), thus increasing the total bound peptide population. Notably, the k_{off} value (2090±230 s⁻¹) was close to that of residues Phe₅₆ and Ile₅₉ found in the two-site exchange analysis, while the apparent R_{2b} of Glu₅₇ decreased (275±50 and 370±65 s⁻¹ at 500 and, 800 MHz, respectively) as compared to that in two-site exchange model (Table 1). Treatment of Glu₅₇ as an independent association-dissociation site, undergoing conformational exchange on the protein surface (FIG. 2, pathway C), yields smaller apparent R_{2b} and larger p_{b}=p_{b1}+p_{b2} (˜5.3%) values, but it is again not fully consistent with the behavior of Phe₅₆ and Ile₅₉ sites since the total bound population of Glu₅₇ becomes larger than the bound populations for the Phe₅₆/Ile₅₉ sites in the two-state analysis. In other words, Glu₅₇ appears to spend more time in “bound” state than Phe₅₆ and Ile₅₉, which is not consistent with the applied model. It is worthwhile to note that in the formal three-state exchange description (FIG. 2, pathway B) the actual separation of “bound” state from “free” state at the residue level is mainly reflected in the large difference between the intrinsic R_{2b} and R_{2f} values. In this respect, although it is easy to imagine a situation when a residue is not interacting (or interacting only weakly) with a prothrombin surface in one of the two bound states in the N-acetyl-Hir(55-65)-prothrombin complex, it would still be in the “bound” state, since its R_{2b} would be closer to that dictated by the correlation time of the complex. Within the limits of this definition, dissociation and association of all residues in the peptide occurs simultaneously, regardless of their residue-specific behavior on the protein surface.

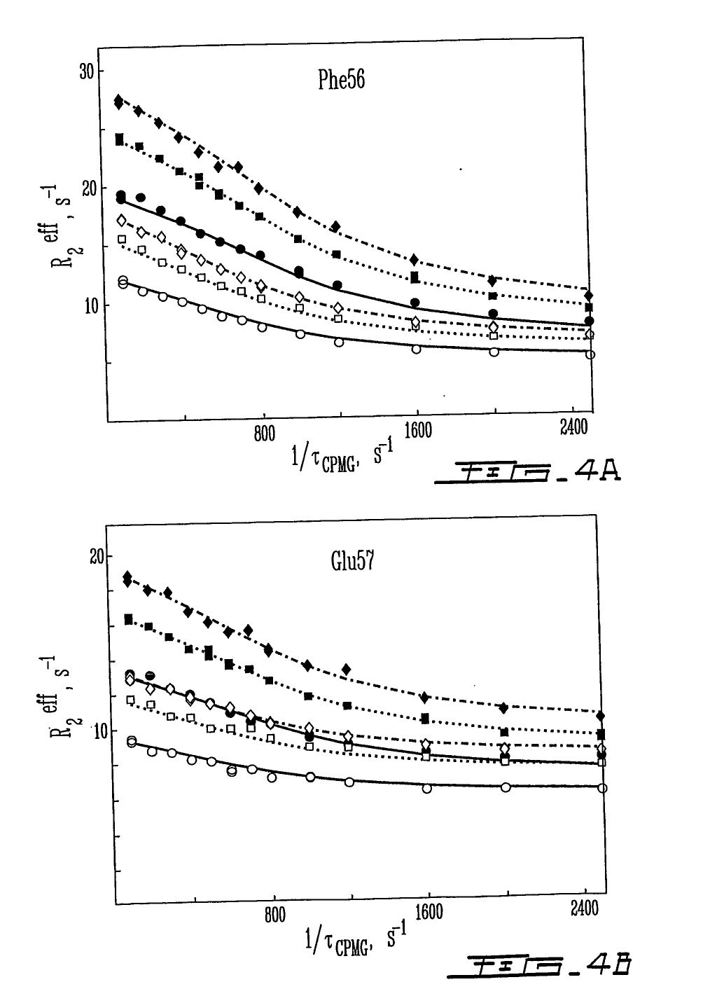

To “link” the process of binding-dissociation between different ¹⁵N sites of the peptide, fitting of all eighteen dispersion curves was performed for the three residues Phe₅₆, Glu₅₇ and Ile₅₉ at three concentrations of prothrombin and at two magnetic fields (FIG. 5A). The experimental curves were in agreement with the “linear” three-site exchange model (FIG. 5A), and yielded k_{off}=2670±140 s⁻¹, k_{off}^{app}=1900±400 s⁻¹, k₁=560±510 s⁻¹, k₂=134±40 s⁻¹, kₑₓ=k₁+k₂=700±500 s¹, p_{b1}=2.30±0.14%, p_{b2}=1.0±0.77%, p_{b}=p_{b1}p_{b2}=3.35±0.75%, R_{2b}=150/200±16/18 s⁻¹ at 500/800 MHz. Here k_{off}^{app}=(p_{b1}×k_{off}¹+p_{b2}×k_{off}²)/(p_{b1}+p_{b2}) is defined as an apparent population-weighted dissociation rate for a general three-state system. In the “linear” three-site exchange model k_{off}² is equal to zero, and the expression for k_{off}^{app} becomes p_{b1}×k_{off}/(p_{b1}+p_{b2}). Overall, the “linear” three-site exchange scheme is consistent with more realistic apparent R_{2b} and p_{b} values, than those found in the two-site exchange scheme. Importantly, the calculated k_{off} value was not strongly compromised upon the addition of the second bound state to the model.

The “forked” three-site mechanism (FIG. 2, pathway D) did not give additional insights in the explanation of experimental results, even if R_{2b} was presumed to be different in the two bound complexes. The fitted curves either did not produce physically meaningful parameters (for example, negative kinetic rate constants and populations were produced), or were similar to the two-site exchange treatment, if k_{off}¹ was assumed to be equal to k_{off}².

In a general three-site exchange mechanism, the presence of two dissociation pathways as well as an exchange between two complex conformers is presumed (FIG. 2, pathway B). Simultaneous fitting of all eighteen dispersion curves for the three residues Phe₅₆, Glu₅₇ and Ile₅₉ at three prothrombin concentrations assuming the same kinetic exchange parameters for every ¹⁵N peak is shown in FIG. 5B. The fitting gave k_{off}¹=2400±1600 s⁻¹, k_{off}²=2600±500 s⁻¹, k_{off}^{app}=2350±230 s⁻¹, k₁=66±57 s⁻¹, k₂=110±130 s⁻¹, kₑₓ=k₁+k₂=180±150 s⁻¹, p_{b1}=1.45±1.0%, p_{b2}1.5±0.5% p_{b}=p_{b1}+p_{b2}=3.0±0.7%, R_{2b}=160/205±26/35 s⁻¹ at 500/800 MHz. The apparent dissociation rate constant k_{off}^{app} has significantly less variability than each of the rate constants k_{off}¹ and k_{off}². It means that fitting of the experimental curves may allow a contribution of a state with k_{off} values markedly different from the average, but its contribution to the total bound species is either small or compensated in the averaged dissociation rate. Some of the fits produced results with k_{off}¹ or k_{off}² close to zero, which essentially corresponds to “linear” mechanism.

In FIGS. 5A and 5B, experimental conditions and labeling are the same as in FIG. 4.

Interestingly, both the “linear” and “full” three-state models failed to describe the relaxation behavior of the backbone ¹⁵N nucleus of Glu₅₈. Although they produce a somewhat closer fit than two-site model, reducing X² from ˜4.8 s⁻² for two-site to ˜1.2 s⁻² for three-site exchange, the calculated parameters are not physically reasonable.

The present invention will be more readily understood by referring to the following examples, which are given to illustrate the invention rather than to limit its scope.

### EXAMPLE 1

### Quantitative Determination of the Dissociation Rate Constant k_{off} of Transient Protein-Ligand Complexes Without Precise Knowledge of the Ligand and Protein Concentrations: Binding of the N-acetyl-Hir(55-65) Peptide to Human Prothrombin

The changes in ¹⁵N transverse relaxation dispersion of a selectively ¹⁵N-labeled N-acetyl-*Asp₅₅-*Phe-*Glu-*Glu-*Ile-Pro₆₀-Glu-Glu-Tyr-Leu-Gln₆₅-COOH (SEQ ID NO:15)(N-acetyl-Hir(55-65)) were followed as a function of the concentration of the binding protein, human prothrombin. The ¹⁵N NMR transverse relaxation rate of the 11-residue peptide is not responsive to the CPMG pulse rate (FIG. 3). Interaction of the peptide with the fibrinogen recognition site of prothrombin (Ni, F., Ning, Q., Jackson, C. M., and Fenton, J. W. (1993) J.Biol.Chem. 268, 16899-16902; and Anderson, P. J., Nesset, A., Dharmawardana, K. R., and Bock, P. E. (2000) J.Biol.Chem. 275, 16428-16434) causes broadening of the peptide's amide proton resonances (Ni, F., Ning, Q., Jackson, C. M., and Fenton, J. W. (1993) J.Biol.Chem. 268, 16899-16902) and the {¹⁵N-¹H}—HSQC peaks, so that the ¹⁵N relaxation dispersion curves can be recorded in the presence of sub-equivalent amounts of the large binding protein. Although the major exchange process in the system was presumed to be the association-dissociation of the peptide-prothrombin complex, it was interesting to see how well a simple two-site exchange model (“bound state-free state”) describes the relaxation behavior of the peptide and whether the dissociation k_{off} rate can be extracted from the experimental NMR relaxation dispersion curves.

In the course of the titration with human prothrombin, each ¹⁵N labeled residue displayed slightly different relaxation behavior in response to increased prothrombin concentrations. Transverse relaxation of residue Asp₅₅ showed a very weak response to the complex formation, implying a small change in chemical shift upon binding. This is in agreement with the very little binding-induced line broadening of the amide proton resonance of Asp₅₅ reported previously (Ni, F., Ning, Q., Jackson, C. M., and Fenton, J. W. (1993) J.Biol.Chem. 268, 16899-16902). Contrary to Asp₅₅, there is a pronounced increase in the transverse relaxation of residues 56-59 induced by binding with prothrombin. Residues Phe₅₆ and Ile₅₉ followed a two-site exchange model fairly well and displayed the same concentration-independent k_{off} and R_{2b} values at both magnetic fields (Table 1). In addition, the calculated bound population for these two residues rose proportionally to the amount of prothrombin added, and were roughly comparable with the absolute concentration of prothrombin. The value of the calculated k_{off} rate was also quite reasonable. One can estimate the K_{D} for the interaction of Hir(55-65) and N-acetyl-Hir(55-65) peptides with human prothrombin to be approximately 100-300 μM (Krstenansky, J. L., Owen, T. J., Yates, M. T., and Mao, S. J. (1987) J.Med.Chem. 30, 1688-1691; and Ni, F., Ning, Q., Jackson, C. M., and Fenton, J. W. (1993) J.Biol.Chem. 268, 16899-16902; and Anderson, P. J., Nesset, A., Dharmawardana, K. R., and Bock, P. E. (2000) J.Biol.Chem. 275, 16428-16434). If one assumes the kₒₙ for N-acetyl-Hir(55-65) to be in the range of 10⁷-10⁸ M⁻¹s⁻¹, as reported for hirudin-based peptides (Skordalakes, E., Elgendy, S., Goodwin, C. A., Green, D., Scully, M. F., Kakkar, V. V., Freyssinet, J. M., Dodson, G., and Deadman, J. J. (1998) Biochemistry 37, 14420-14427; and Myles, T., Le Bonniec, B. F., Betz, A., and Stone, S. R. (2001) Biochemistry 40, 4972-4979; and Betz, A., Hofsteenge, J., and Stone, S. R. (1991) Biochem.J. 275 (Pt 3), 801-803), k_{off}=K_{D}kₒₙ can be estimated as 10³-10⁴ s⁻¹, in good agreement with the k_{off} value determined here by use of ¹⁵N NMR relaxation dispersion spectroscopy (Table 1).

Residues Glu₅₇ and Glu₅₈ displayed a complex NMR relaxation behavior suggesting the presence of other binding states. The most obvious deviation from a simple two-state binding was found for Glu₅₈. The transverse relaxation of Glu₅₈ was growing with the addition of prothrombin too slowly to be consistent with either two-state or even three-state exchange models, although the three-state model represented a slightly better description. Although the relaxation dispersion curves of Glu₅₇ were possible to be fitted to a two-state exchange model, the apparent values of R_{2b} for Glu₅₇ were significantly higher, whereas p_{b} was lower than those of Phe₅₆ and Ile₅₉. To establish if extension of the exchange model to a three-state process will produce more realistic values of R_{2b} and p_{b}, the relaxation of the peptide was analyzed at all three prothrombin concentrations simultaneously. Since the rate constants of the conformational exchange between the bound states do not depend on the prothrombin concentration, whereas the pseudo-first order binding rate constant is proportional to [E_{f}], titration by the binding protein may in principle help isolate the contributions of ligand association-dissociation and conformational change of the protein-ligand complex to the observed transverse relaxation. The fitted k₁ and k₂ rates for the conformational change were on the order of 100 s⁻¹ for the prothrombin-peptide complex which is comparable with those (˜115 and ˜185 s⁻¹, respectively) obtained with the stopped-flow fluorescence spectroscopy for thrombin-peptide interactions (Jackman, M. P., Parry, M. A., Hofsteenge, J., and Stone, S. R. (1992) J.Biol.Chem. 267, 15375-15383). At the same time, R_{2b} and p_{b} values turned out to be closer to the expected physically meaningful values, than those obtained by a two-state exchange scheme. It appears that the k_{off} value obtained from the “linear” three-site exchange scheme might be underestimated by a two-site model, but not dramatically.

The contributions of multi-site exchanges or conformational changes of the protein-ligand complex can be assessed by a full three-state exchange scheme (FIG. 2, pathway B). Similarly to the “linear” three-site exchange scheme, full three-state exchange scheme (FIG. 2, pathway B) reduces R_{2b} closer to physically reasonable values. The k_{off} values for both bound states display larger variability than in the schemes with only one dissociation-competent species. It was, however, noticed that a state with k_{off} rate constant significantly larger or smaller, than the average, might have only small contribution to the total bound population. The apparent k_{off} rate constant has much smaller variation, which appears to be the consequence of the restraints imposed by the transverse relaxation dependence on the protein concentration. Overall, the full three-state exchange scheme did not explain the increased apparent R_{2b} values any better than the “linear” three-state exchange scheme. The apparent dissociation rate constant k_{off}^{app} is again rather close to the value obtained for residues Glu₅₇ and Ile₅₉ by means of a two-state exchange mechanism.

In summary, the dissociation rate k_{off} can be obtained by fitting a two-state exchange model to the ¹⁵N relaxation dispersion curves obtained for the N-acetyl-Hir(55-65) peptide at two external magnetic fields and at three prothrombin concentrations (FIG. 4 and Table 1). The accuracy of the absolute peptide or prothrombin concentrations was not critical, as only the increases in the prothrombin/peptide ratio, measured by the volume of the added prothrombin stock solution, need to be included in the data fitting process. This approach in addition makes it possible to separate ligand sites (i.e. residues Phe₅₆ and Ile₅₉) obeying a two-state binding mechanism (including one free and one bound ligand states) from more complex exchange mechanisms, including one free and two or more bound ligand states (i.e. residues Glu₅₇ and Glu₅₈). The nuclei following a two-state binding mechanism behave similarly to each other and display reasonable physical parameters, such as k_{off}, p_{b}, R_{2b}(500), R_{2b}(800), and δω_{bf}. The derived k_{off} value is independent of protein concentration and can serve as a measure of the binding affinity of a transient protein-ligand complex.

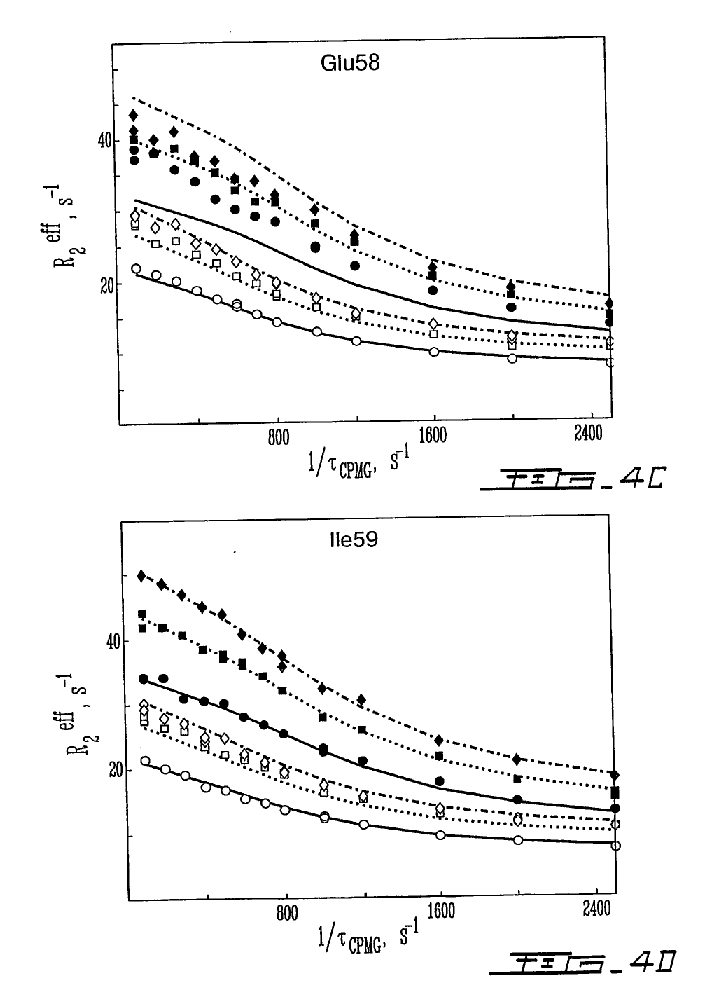

The different binding behavior of ligand sites can be further verified by the temperature dependence of the dissociation rate constants of a protein-ligand complex. The peptide ¹⁵N relaxation dispersion was collected at three different temperatures for the N-acetyl-Hir(55-65) peptide in complex with human prothrombin (Table 2 and FIG. 6), with an approximate prothrombin:peptide ratio of ˜1:30. The k_{off} values of the ¹⁵N sites obeying a two-state binding behavior, namely Phe₅₆ and Ile₅₉, grow monotonously and are identical at each temperature within the experimental error. The apparent dissociation rates for the ¹⁵N sites obeying a more complex exchange behavior (i.e. residues Glu₅₇ and Glu₅₈) differ from each other and from other ¹⁵N sites, and have very different temperature dependence from two-state binding sites. For ligand sites obeying two-state binding, temperature dependence of the k_{off} rates can be used to derive quantitative information on the energetic barriers of the complex dissociation, including the enthalpy and entropy of activation (Jardetzky, O. and Roberts, G. C. K. (1981) NMR in molecular biology. New York: Academic Press; and Sandstrom, J. (1982) Dynamic NMR spectroscopy. London: Academic Press). These energetic parameters can be used to assess the origins of fast dissociation for transient protein-ligand complexes, providing the physico-chemical basis for affinity enhancement and optimization.

In FIG. 6, the peptide/protein molar ratio is approximately 30:1. Calculation of the apparent k_{off} was performed using a two-state exchange approximation. Other experimental conditions are as in FIG. 4.

TABLE 2
Kinetic and ¹⁵N Relaxation Dispersion Parameters of the N-acetyl-Hir(55-65)
Peptide in Complex with Human Prothrombin for a Two-Site Exchange at
different temperatures (protein:peptide ratio 1:30)

| Residue/T | T, K | k_{off}, s⁻¹ | δω_{bf}, ppm | p_{b}, % | R_{2b} [R_{2b}(800)], s⁻¹ | |
|---|---|---|---|---|---|---|
| Phe56 | 298 | 2150 ± 100 | 4.2 ± 0.2 | 2.53 ± 0.15 | 190 ± 7 | [303 ± 18] |
| Phe56 | 288 | 1280 ± 50 | 3.4 ± 0.1 | 4.18 ± 0.15 | 150 ± 5 | [226 ± 9] |
| Phe56 | 278 | 930 ± 40 | 3.1 ± 0.1 | 4.92 ± 0.17 | 171 ± 5 | [257 ± 8] |
| Glu57 | 298 | 2030 ± 200 | −3.5 ± 0.4 | 1.92 ± 0.21 | 400 ± 40 | [513 ± 58] |
| Glu57 | 288 | 1200 ± 90 | −1.9 ± 0.2 | 5.02 ± 0.21 | 194 ± 26 | [238 ± 34] |
| Glu57 | 278 | 500 ± 50 | −1.5 ± 0.1 | 7.18 ± 0.45 | 201 ± 13 | [259 ± 19] |
| Glu58 | 298 | 1700 ± 80 | 5.4 ± 0.2 | 3.32 ± 0.15 | 259 ± 8 | [367 ± 15] |
| Glu58 | 288 | 690 ± 50 | 5.0 ± 0.1 | 5.57 ± 0.29 | 195 ± 8 | [271 ± 11] |
| Glu58 | 278 | 1030 ± 50 | 4.2 ± 0.1 | 5.27 ± 0.19 | 214 ± 6 | [294 ± 8] |
| Ile59 | 298 | 2260 ± 100 | −4.5 ± 0.2 | 4.41 ± 0.28 | 193 ± 8 | [312 ± 18] |
| Ile59 | 288 | 1170 ± 50 | −4.0 ± 0.1 | 6.01 ± 0.20 | 189 ± 5 | [280 ± 10] |
| Ile59 | 278 | 1020 ± 50 | −3.6 ± 0.1 | 5.86 ± 0.22 | 260 ± 9 | [345 ± 12] |

### EXAMPLE 2

### Quantitative Determination of the Dissociation Rate Constant k_{off} of Transient Protein-Ligand Complexes Without Precise Knowledge of the Ligand and Protein Concentrations: a Uniformly s⁵N-Labeled Recombinant Peptide Interacting With Human Prothrombin

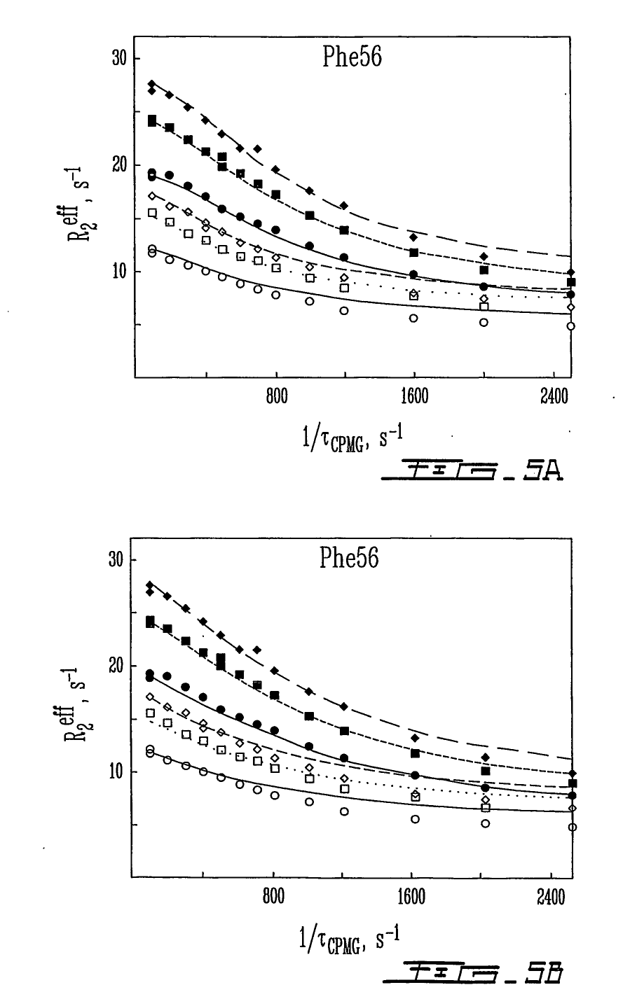

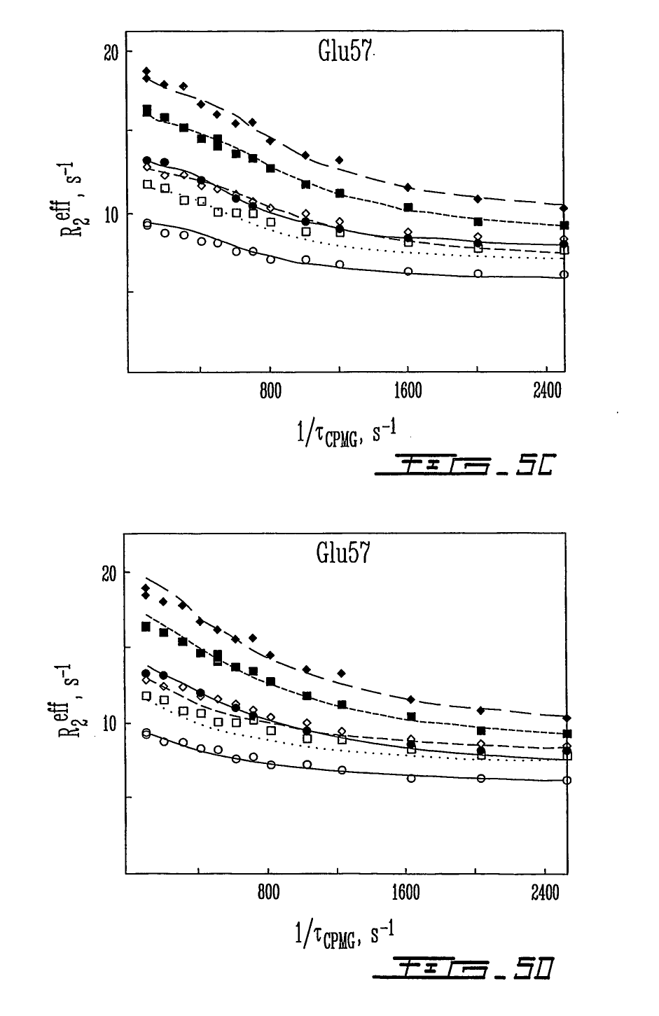

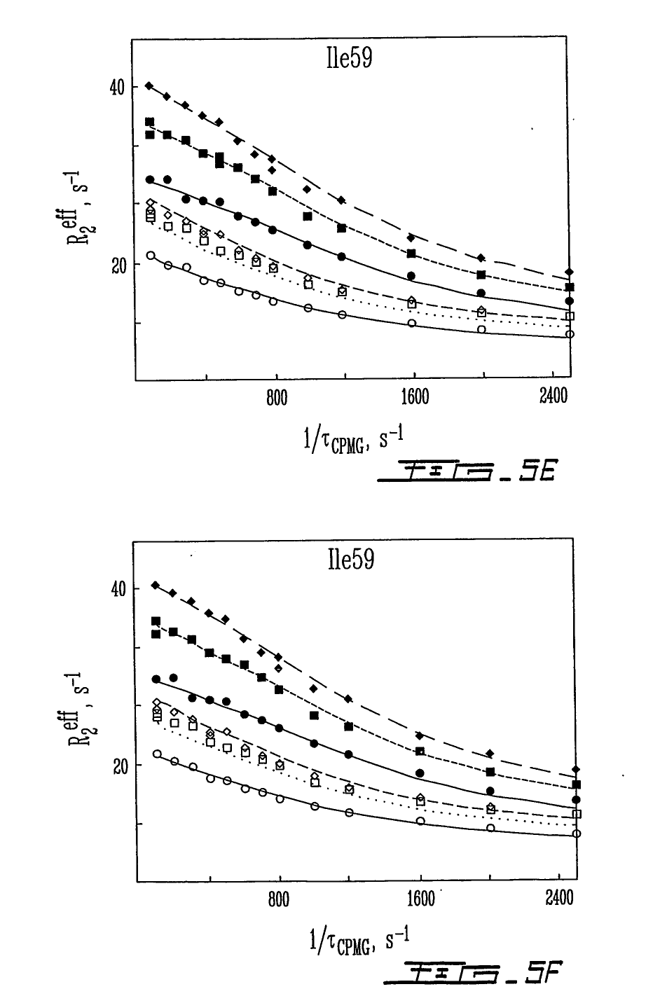

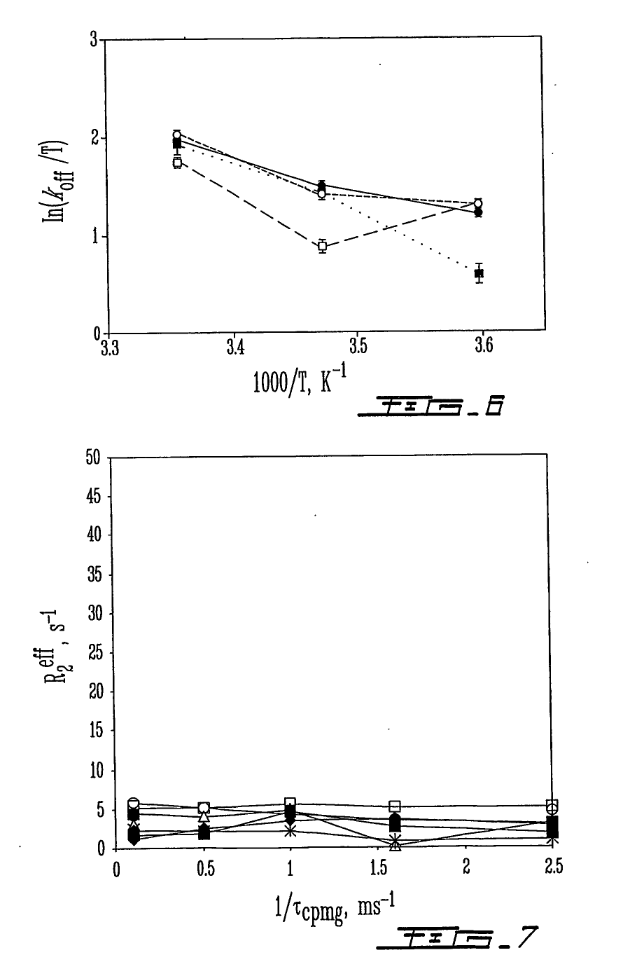

A peptide Gly₅₄-Asp₅₅-Phe₅₆Glu₅₇-Glu₅₈-Ile₅₉-Pro₆₀-Glu₆₁-Glu₆₂-Tyr₆₃-Leu₆₄-Gln₆₅ (SEQ ID NO:19)(Hir(54-65)), related to the N-acetyl-Hir(55-65) peptide, was recombinantly expressed and uniformly labeled with the ¹⁵N isotope. The Hir(54-65) peptide was dissolved at ˜1.5 mM in an aqueous solution that was 50 mM in sodium phosphate at pH 5.5. NMR peak assignments were carried out as described hereinabove. The free peptide produces relaxation dispersion curves independent of the CPMG pulse rate (FIG. 7). Upon the addition of prothrombin, the peptide gives ¹⁵N relaxation dispersion curves and k_{off} rates very similar to those obtained for its synthetic analog N-acetyl-Hir(55-65) (FIGS. 4 and 8, Table 3).

In FIG. 7, the curves for every backbone ¹⁵N site are flat and display no apparent CPMG pulse rate dependence. The peptide was ˜1.0 mM in an aqueous solution (10% D₂O) that was 50 mM in sodium phosphate at pH 5.5.

TABLE 3
Kinetic and ¹⁵N Relaxation Dispersion Parameters of the
Recombinant Hir(54-65)-Peptide in Complex with Human
Prothrombin for a Two-Site Exchange Scheme at 288 K.

| | k_{off} (s⁻¹) | δω_{bf}, ppm | p_{b}, % | R_{2b} [R_{2b}(800)] (s⁻¹) | | R_{2f} [R_{2f}(800)] (s⁻¹) |
|---|---|---|---|---|---|---|
| Phe56 | 980 ± 50 | 4.3 ± 0.1 | 4.5 ± 0.1 | 200 ± 5 | [242 ± 8] | 1.69 [2.88] |
| Glu57 | 990 ± 80 | 2.2 ± 0.2 | 4.9 ± 0.3 | 265 ± 20 | [310 ± 30] | 2.39 [2.40] |
| Glu58 | 610 ± 45 | 4.7 ± 0.1 | 6.5 ± 0.4 | 232 ± 10 | [214 ± 10] | 1.97 [4.38] |
| Ile59 | 550 ± 60 | 5.5 ± 0.2 | 6.0 ± 0.5 | 270 ± 20 | [300 ± 20] | 3.65 [5.27] |
| Glu62 | 740 ± 60 | 1.8 ± 0.1 | 6.9 ± 0.5 | 140 ± 9 | [193 ± 13] | 3.61 [3.81] |
| Tyr63 | 980 ± 45 | 2.9 ± 0.1 | 6.7 ± 0.3 | 163 ± 6 | [263 ± 12] | 3.69 [2.85] |

In FIGS. 8A to 8F, the dispersion curves were recorded at two 15N frequencies, 50.684 MHz (◯) and 81.076 MHz (●). Experimental values for every residue were fitted to a two-site model. Other experimental conditions are as in FIG. 7.

### EXAMPLE 3

### Quantitative Determination of the Dissociation Rate Constant k_{off} of Transient Protein-Ligand Complexes Without Precise Knowledge of the Ligand and Protein Concentrations: the Synthetic N-acetyl-Hir(55-65) Peptide in Complex With Human Thrombin

The peptide N-acetyl-Hir(55-65) also binds to human thrombin with a higher affinity than for the same site on human prothrombin (Ni, F., Ning, Q., Jackson, C. M., and Fenton, J. W. (1993) J.Biol.Chem. 268, 16899-16902).

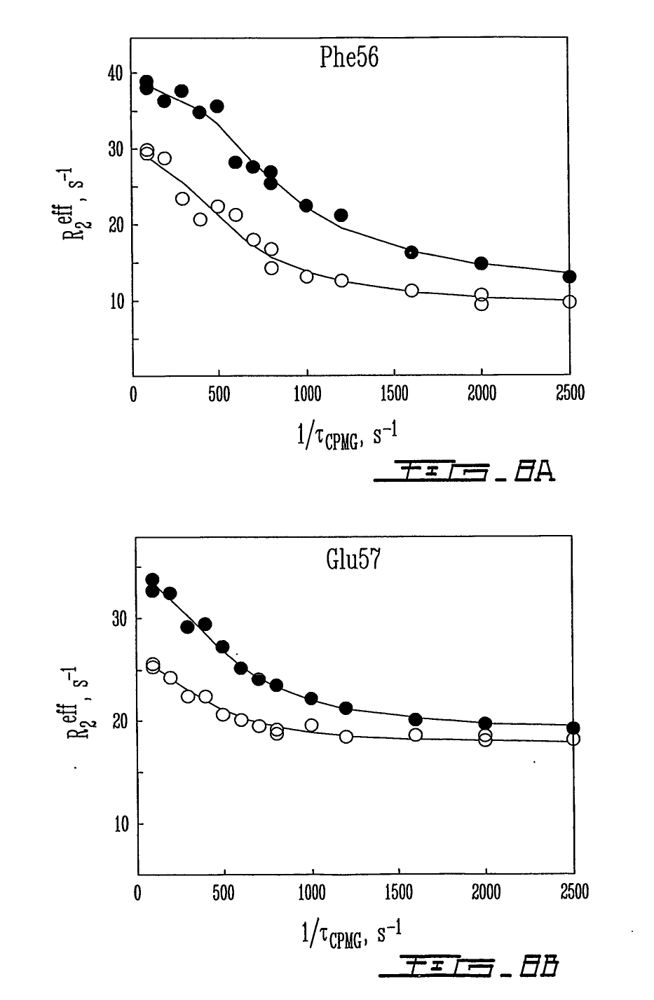

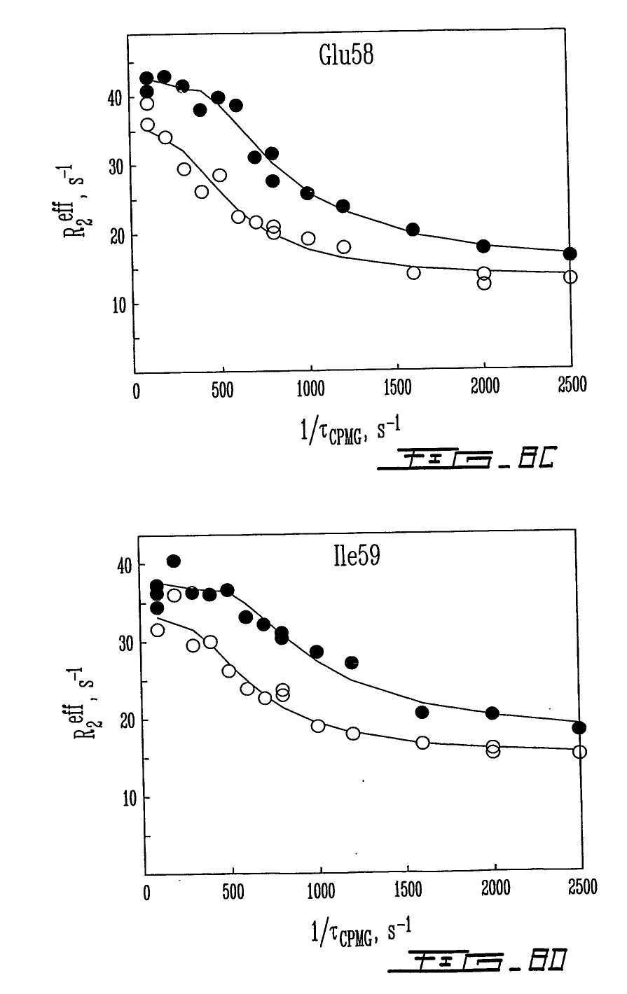

FIGS. 9A to 9D show the ¹⁵N relaxation dispersion profiles for the residues of the peptide N-acetyl-Hir(55-65) in the presence of human thrombin at a protein:peptide ratio of ˜1:15. The dispersion curves show expected quantitative differences compared to those for the N-acetyl-Hir(55-65)-prothrombin complex. There were little differences in the lineshapes of the one-dimensional proton NMR spectra of the thrombin-peptide complex obtained at the two magnetic fields, namely at 500 and 800 MHz. On the other hand, the one-dimensional proton NMR spectra of the prothrombin-peptide complex were significantly more broadened at the higher magnetic field. This field dependence of the proton spectral lineshapes already indicate an intermediate to slow exchange situation for the thrombin-peptide complex. Fitting of the dispersion curves indeed shows a decreased dissociation rate for the thrombin-peptide complex (Table 4). Residue Phe₅₆ shows an elevated apparent k_{off} (˜650 s⁻¹), unreasonably large R_{2b} at 800 MHz, and small p_{b} values, which indicates that it may be involved in additional conformational exchange processes. Therefore, comparison of k_{off} values for the N-acetyl-Hir(55-65)-thrombin complex and N-acetyl-Hir(55-65)-prothrombin complex reveal an expected acceleration for the dissociation of the weaker N-acetyl-Hir(55-65)-prothrombin complex.

TABLE 4
Kinetic and ¹⁵N Relaxation Dispersion Parameters of the N-acetyl-Hir(55-65)
Peptide in Complex with Human α-Thrombin for a Two-Site Exchange Scheme
(protein:peptide ratio ˜1:15) at 298 K.¹

| | k_{off} (s⁻¹) | δω_{bf}, ppm | p_{b}, % | R_{2b} [R_{2b}(800)] (s⁻¹) | | R_{2f} [R_{2f}(800)] (s⁻¹) |
|---|---|---|---|---|---|---|
| Phe56 | 645 ± 69 | 2.5 ± 0.2 | 1.28 ± 0.12 | 54 ± 10 | [276 ± 23] | 1.38 [0.95] |
| Phe56* | 628 ± 57 | 1.5 ± 0.1 | 1.3 ± 0.08 | 61 ± 5 | [264 ± 17] | |
| Glu57 | 266 ± 49 | −1.6 ± 0.1 | 2.49 ± 0.35 | 108 ± 12 | [81 ± 8] | 1.60 [1.44] |
| Glu57* | 279 ± 55 | −0.9 ± 0.1 | 2.4 ± 0.3 | 106 ± 10 | [79 ± 7] | |
| Glu58 | 217 ± 69 | 3.5 ± 0.3 | 2.97 ± 1.36 | 134 ± 33 | [58 ± 20] | 2.14 [2.53] |
| Glu58* | 213 ± 85 | 2.1 ± 0.1 | 3.4 ± 2.6 | 126 ± 40 | [56 ± 20] | |
| Ile59 | 134 ± 45 | −4.5 ± 0.3 | 5.9 ± 1.8 | 200 ± 50 | [88 ± 26] | 0.71 [1.03] |
| Ile59* | 104 ± 60 | −2.7 ± 0.1 | 6.7 ± 4.4 | 132 ± 67 | [60 ± 30] | |

¹Rows denoted with asterisk show the results of the fitting with a single-exponential approximation.

In FIGS. 9A to 9D, the concentration of the peptide was ˜2 mM with a concentration ratio of ˜15:1 for the peptide and thrombin. For the dispersion profiles at 500 MHz, a total of 15 HSQC spectra were recorded with effective B1 fields (or 1/τCPMG) of 50, 100, 150, 200, 250, 300, 400, 500, 600, 700, 800, 1000, 1200, 1600 and 2000 Hz. For the dispersion profiles at 800 MHz, a total of 15 HSQC spectra were recorded with effective B₁ fields of 50, 100(2),150, 200, 250, 300,400, 500, 600, 700, 800, 1000(2), and 1200 Hz, of which the experiments were repeated twice for the 100 and 1000 Hz points. The total length of the ¹⁵N CPMG pulse train, T (FIG. 1), was set to 80 ms, 53.336 ms and 48 ms for the CPMG fields of 50, 150 and 250 Hz, respectively, and kept at a constant value of 40 ms for the rest of the experiments. The total number (4N) of ¹⁵N CPMG 180° pulses was 48 at 800 MHz and 80 at 500 MHz. As with FIG. 4, the calculated curves based on the fitted parameters (Table 4) are plotted as solid lines for the amide ¹⁵N resonances of residues Phe56 (FIG. 9A), Glu57 (FIG. 9B), Glu58 (FIG. 9C) and Ile59 (FIG. 9D).

### EXAMPLE 4

### Quantitative k_{off} Determination With Ligand Mixtures: the Synthetic N-acetyl-Hir(55-65) and a Mixture of Peptides Targeting Different Binding Sites on Human Thrombin

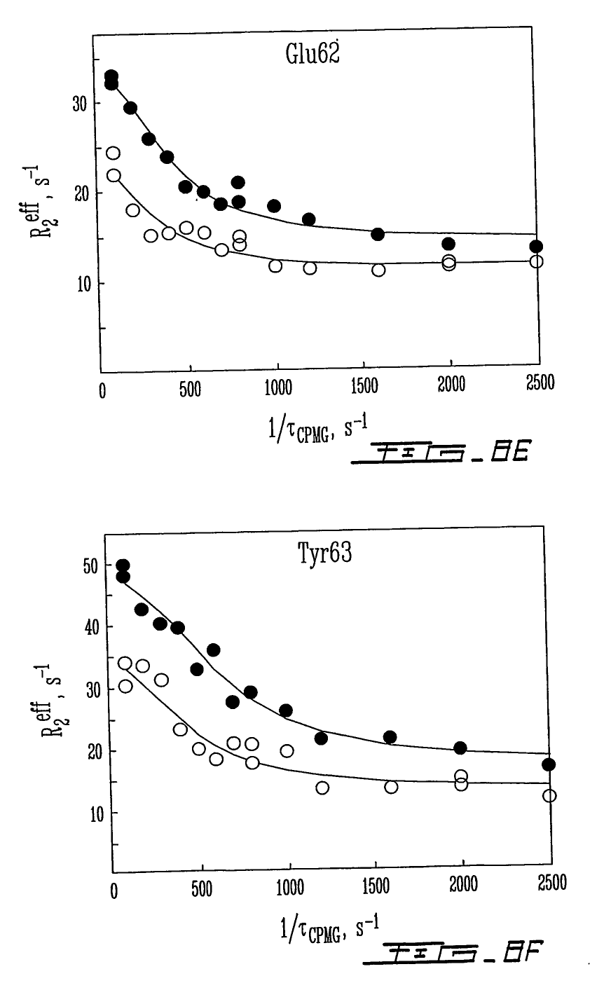

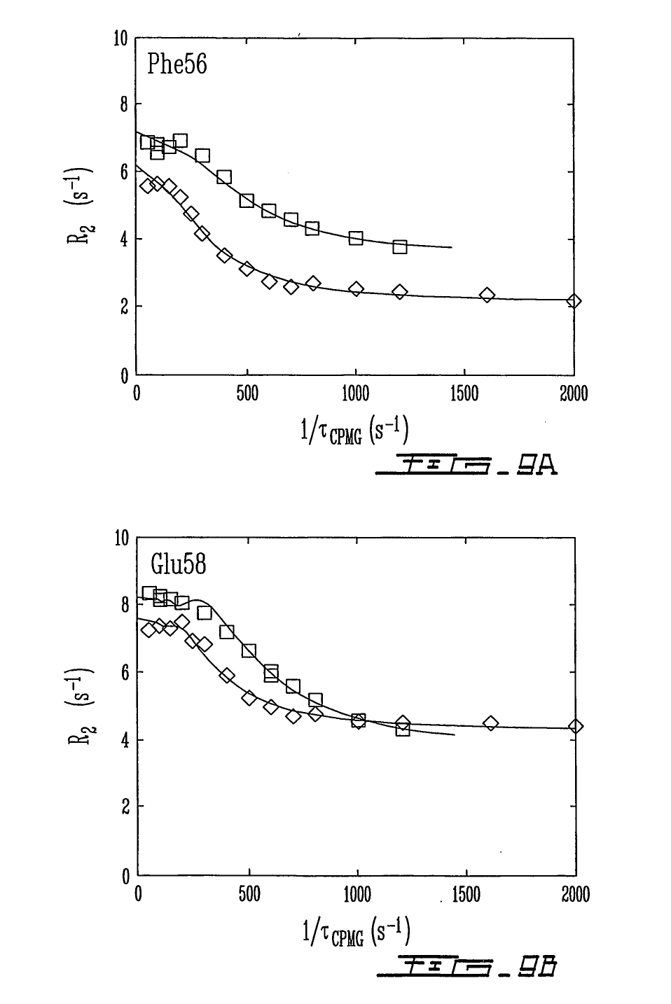

A special procedure was used to express six uniformly ¹⁵N-labeled hexa/penta-peptides, GLDPRH_{L} (SEQ ID NO:1), GVDPRH_{L} (SEQ ID NO:2), GFNPRH_{L} (SEQ ID NO:3), GPNPRH_{L} (SEQ ID NO:4), GFSARH_{L} (SEQ ID NO:5), and GVSPR (SEQ ID NO:6), where a one-letter code is used to define the amino acid sequence, and H_{L} stands for homoserine lactone. The six short peptides were expressed in tandem as a linear sequence GLDPRMGVDPRMGFNPRMGPNPRMGFSARMGVSPR (SEQ ID NO:16). The individual peptides were released by CNBr cleavage at the methionine residues, producing the homoserine lactone (H_{L}) derivatives of the pentapeptides. Upon introduction of the hexa/pentapeptides in the sample of the N-acetyl-Hir(55-65)-thrombin complex (see Example 3), the five ¹H—¹⁵N HSQC cross-peaks of the N-acetyl-Hir(55-65) peptide were still easily resolved and identifiable (FIGS. 10A and 10B). The N-acetyl-Hir(55-65) peptide and the hexa/pentapeptides target the anion-binding exosite and the catalytic active site of human thrombin, respectively. The hexa/pentapeptides are proteolytically cleaved after the arginine residues in the presence of the human thrombin to produce the GLDPR (SEQ ID NO:7), GVDPR (SEQ ID NO:8), GFNPR (SEQ ID NO:9), GPNPR (SEQ ID NO:10), GFSAR (SEQ ID NO:11), and GVSPR (SEQ ID NO:6) pentapeptides targeting the thrombin active site. In FIGS. 10A and 10B, NMR spectra are recorded in 50 mM sodium phosphate buffer (10% D₂O), pH 5.5, at 288K, and at 800 MHz. The ¹⁵N-labeled residues of the N-acetyl-Hir(55-65) peptide are well resolved and assigned. Arrows 1 to 4 indicate some of the unassigned [¹H—¹⁵N]—HSQC cross-peaks of the pentapeptides GLDPR (SEQ ID NO:7), GVDPR (SEQ ID NO:8), GFNPR (SEQ ID NO:9), GPNPR (SEQ ID NO:10), GFSAR (SEQ ID NO:11), and GVSPR (SEQ ID NO:6). ¹⁵N relaxation dispersion curves of the N-acetyl-Hir(55-65) peptide (FIG. 11A) and some resonances of the pentapeptides (FIG. 11B) can still be recorded, which demonstrate that k_{off} values for the N-acetyl-Hir(55-65) peptide/human α-thrombin complex can be derived in the presence of other peptides. FIG. 11 A represents ¹⁵N relaxation dispersion curves displayed by residues Asp55 (◯), Phe56 ( [symbol] ), Glu57 (□), Glu58 (⋄) and Ile59 (Δ) of the N-acetyl-Hir(55-65) peptide. FIG. 11B represents relaxation dispersion curves of peaks 1 ( [symbol] ), 2 (●), 3 (▪), and 4 (♦) (FIG. 10B) of the pentapeptides. Other experimental conditions are as in FIGS. 10A and 10B.

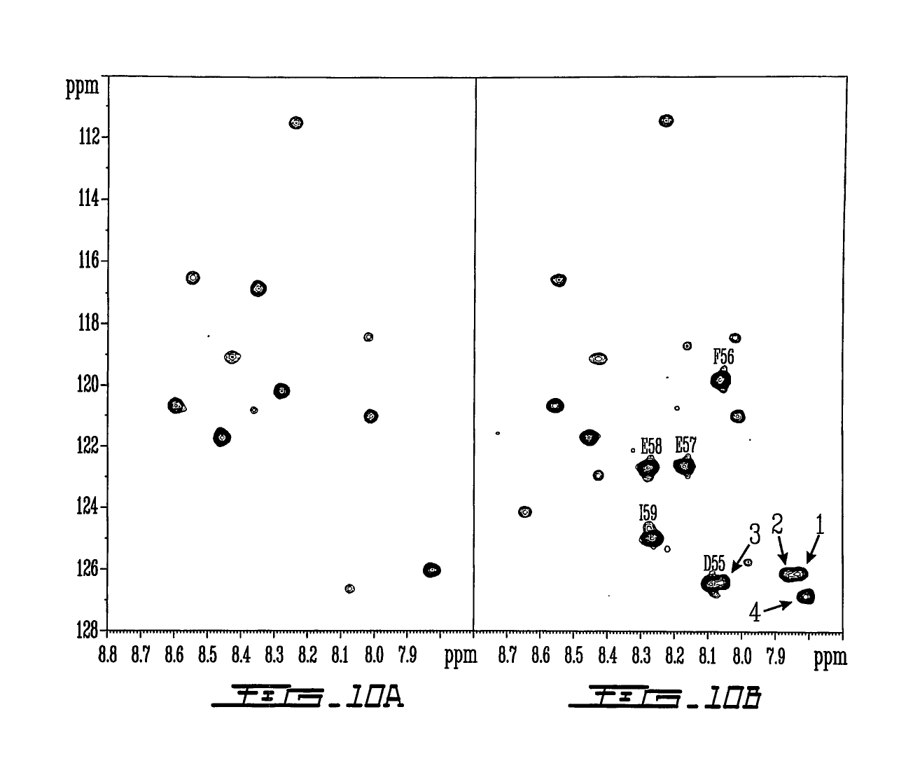

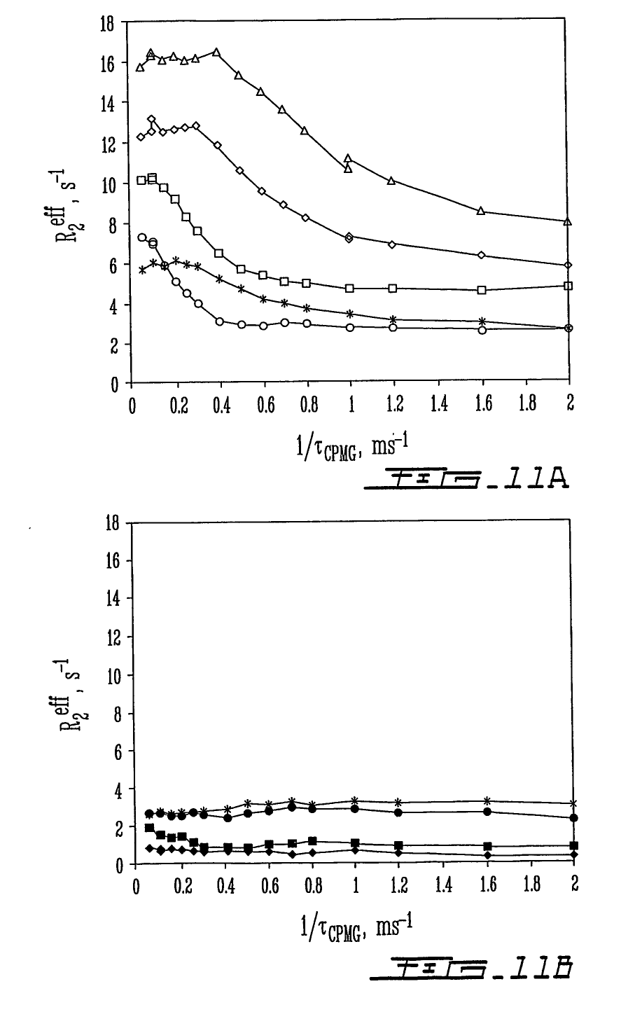

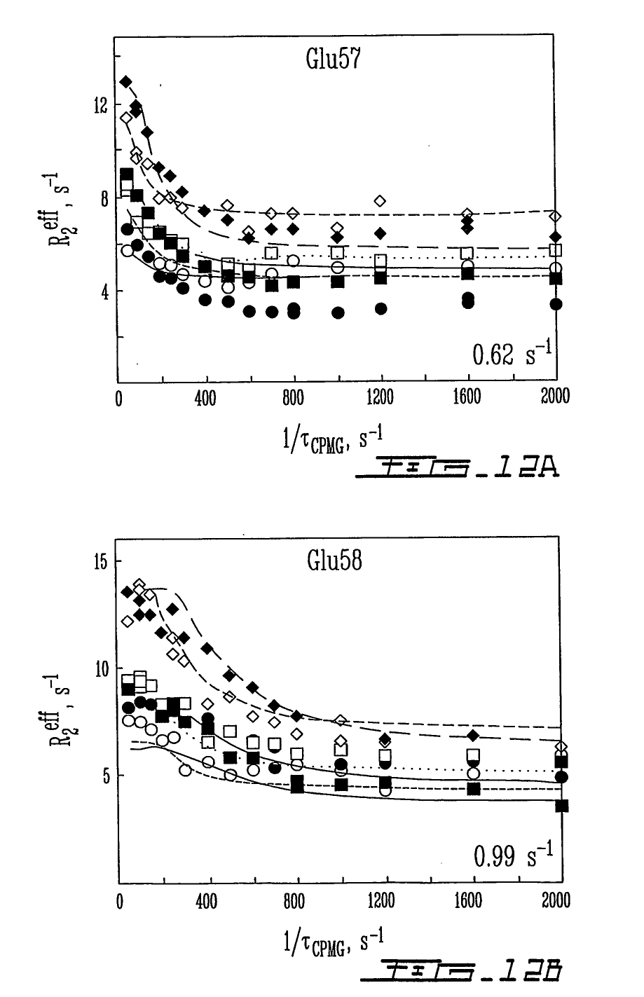

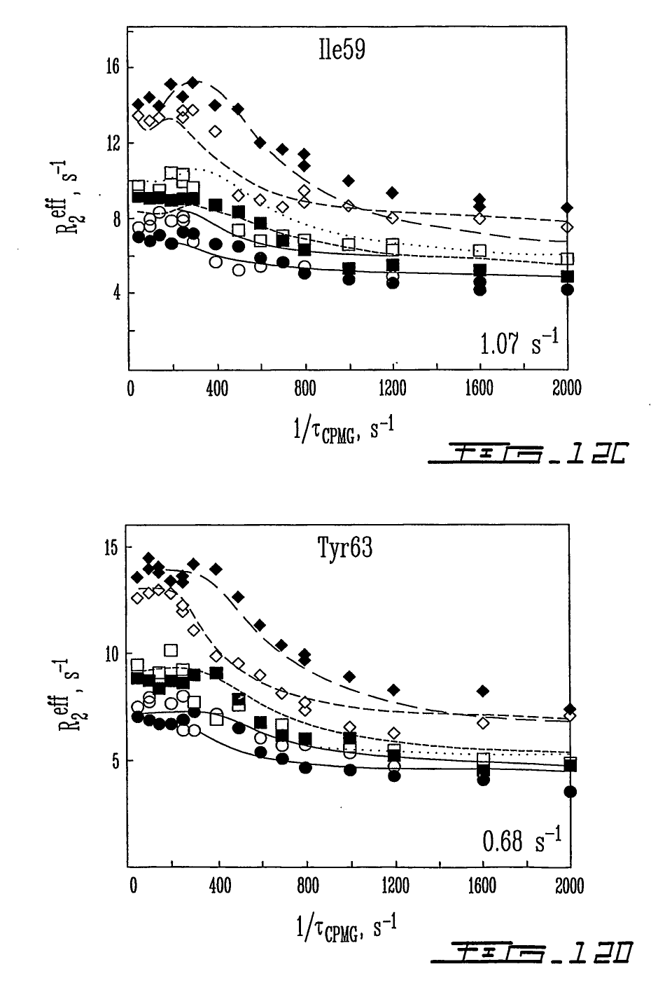

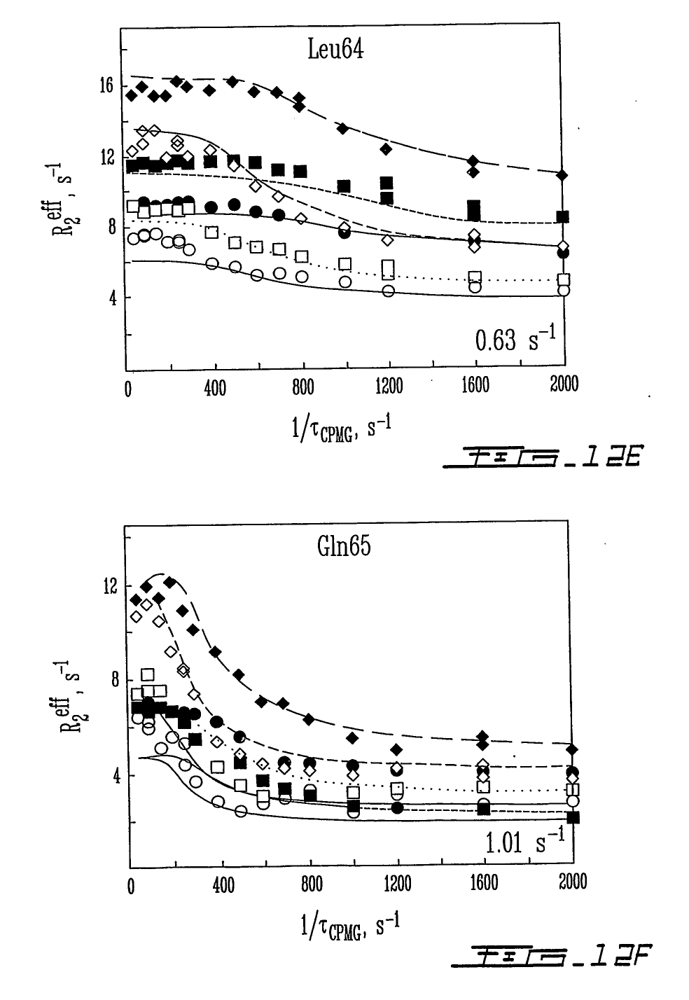

Quantitative k_{off} determination was carried out for the mixture of a short peptide Phe-Asp₄₅-Pro-Arg (FD22-N)(SEQ ID NO:17) with Pro-Gln-Ser₅₀-His-Asn-Asp-Gly-Asp₅₅-Phe-Glu-Glu-Ile-Pro₆₀-Glu-Glu-Tyr-Leu-Gln₆₅ (FD22-C) (SEQ ID NO:18), which contain the sequence of the N-acetyl-Hir(55-65) peptide. Calculated apparent k_{off} values for the peptides FD22-N and FD22-C are presented in Table 5. Experimental values for every residue (FIGS. 12A to 12H), except for Phe₅₆ and Arg₄₇, were fitted to a two-site model using kₒₙ′, 1.52×kₒₙ′, and 2.78×kₒₙ′ for the pseudo-first-order association rate constants. Residues Phe₅₆ and Arg₄₇ displayed very little response to the CPMG pulse rate at the two lower thrombin concentrations, and were therefore fitted to a two-state exchange scheme using the dispersion data obtained at the highest thrombin concentration. Residue Tyr63 obey well the two-site exchange mechanism within the error of the experiment, with k_{off} being approximately equal to 100 s⁻¹. Residues Phe₅₆ and Leu₆₄ display the most profound deviation from the two-state exchange behavior. They show elevated apparent k_{off} values (˜400 s⁻¹), unreasonably large R_{2b} and small p_{b} values. This behavior is typical of residues experiencing extensive conformational exchange in the bound state. Observation of relaxation dispersion for the Arg₄₇ residue of the FD22-N peptide confirms the possibility to measure k_{off} rates for a number of peptides targeting the same protein non-competitively.

In FIGS. 12A to 12H, the dispersion curves were recorded at two ¹⁵N frequencies, 50.684 MHz (◯,●, and ⋄) and 81.076 MHz (●,▪, and ♦), and at three increasing thrombin:peptide ratios. Experimental values for every residue, except for Phe₅₆ and Arg₄₇, were fitted to a two-site model using kₒₙ′ (◯, ●, solid lines), 1.52×kₒₙ′ (□, ▪, dotted lines), and 2.78×kₒₙ′ (⋄, ♦, dash-dot lines) for the pseudo-first-order association rate constants at these thrombin concentrations. Residues Phe₅₆ and Arg₄₇ displayed very little response to the CPMG pulse rate at the two lower thrombin concentrations, and were therefore fitted to a two-state exchange scheme using the dispersion data obtained at the highest thrombin concentration. Open and filled circles for Phe₅₆ and Arg₄₇ correspond to data obtained at 50.684 MHz and 81.076 MHz, respectively. Values shown at the right lower corners represent r.m.s.d. of the fitted curves with respect to the experimental values.

TABLE 5
Kinetic and ¹⁵N Relaxation Dispersion Parameters of the thrombin-cleaved FD22-Peptide
in Complex with Human Thrombin for a Two-Site Exchange
Scheme at 288 K.¹

| | k_{off} (s⁻¹) | δω_{bf}, ppm | p_{b}, % | R_{2b} [R_{2b}(800)] (s⁻¹) | | R_{2f} [R_{2f}(800)] (s⁻¹) |
|---|---|---|---|---|---|---|
| Phe56 | 390 ± 40 | 3.0 ± 0.1 | 3.3 ± 0.3 | 170 ± 10 | [330 ± 20] | 2.15 [2.46] |
| Glu57 | 106 ± 110 | 1.1 ± 0.2 | 5.0 ± 1.2 | 84 ± 7 | [31 ± 2] | 2.86 [3.67] |
| Glu58 | 107 ± 23 | 3.0 ± 0.1 | 3.9 ± 0.8 | 92 ± 12 | [79 ± 10] | 2.52 [2.12] |
| Ile59 | 53 ± 27 | 4.9 ± 0.5 | 7.9 ± 3.8 | 84 ± 24 | [18 ± 5] | 3.23 [4.96] |
| Tyr63 | 103 ± 11 | 4.2 ± 0.1 | 3.5 ± 0.4 | 79 ± 6 | [48 ± 4] | 2.93 [3.49] |
| Leu64 | 394 ± 54 | 6.1 ± 0.1 | 1.1 ± 0.2 | 208 ± 21 | [294 ± 30] | 2.06 [4.50] |
| Gln65 | 85 ± 14 | 2.7 ± 0.1 | 4.9 ± 0.7 | 52 ± 5 | [70 ± 7] | 0.57 [0.63] |
| Arg47 | 310 ± 40 | 1.3 ± 0.2 | 3.2 ± 0.4 | 110 ± 10 | [145 ± 20] | 1.0 [1.2] |
| ¹Every residue, except for Phe₅₆ and Arg₄₇, lists parameters obtained by a simultaneous fit to dispersion data at three α-thrombin concentrations using kₒₙ′, 1.52 × kₒₙ′, and 2.78 × kₒₙ′ for the pseudo-first-order association rate constants. Values in p_{b} column for these residues correspond to | | | | | | |
| # the lowest thrombin:ligand ratio. Residues Phe₅₆ and Arg₄₇ displayed very little response to the CPMG pulse rate at the two lower α-thrombin concentrations, and were fitted to a two-state exchange scheme at the highest α-thrombin concentration. Values in p_{b} column for the residues Phe₅₆ and Arg₄₇ correspond to the highest thrombin:ligand ratio. | | | | | | |

### EXAMPLE 5

### Quantitative k_{off} Determination With Ligand Mixtures: Identification of Cooperative Effects Between Two Peptides Targeting Distinctive Sites On the Cdc42 Protein from Candida Albicans

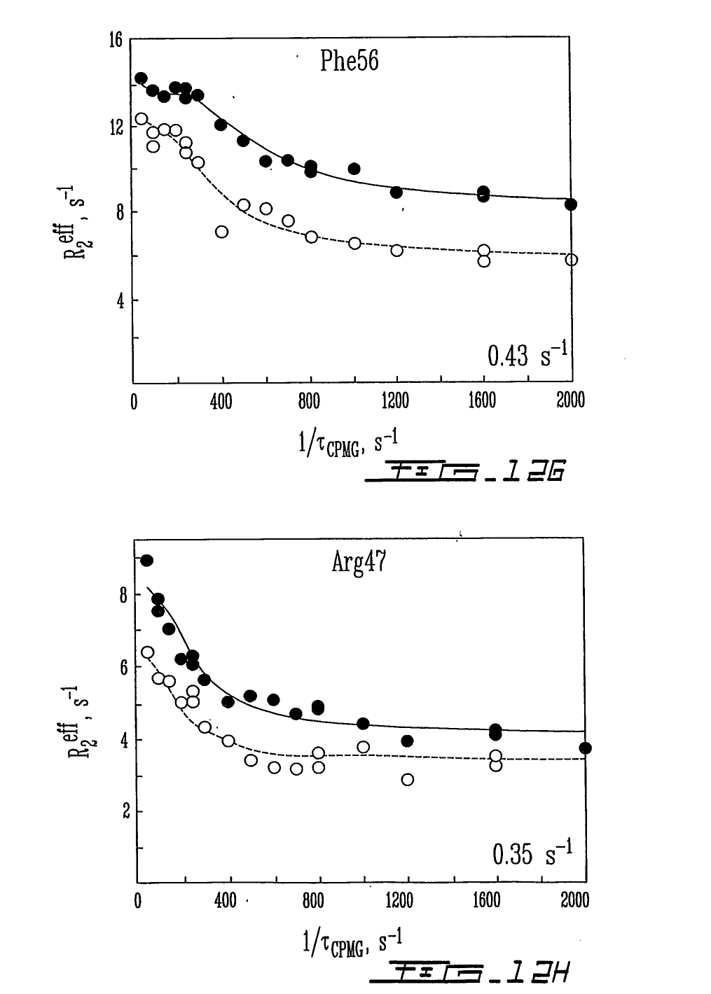

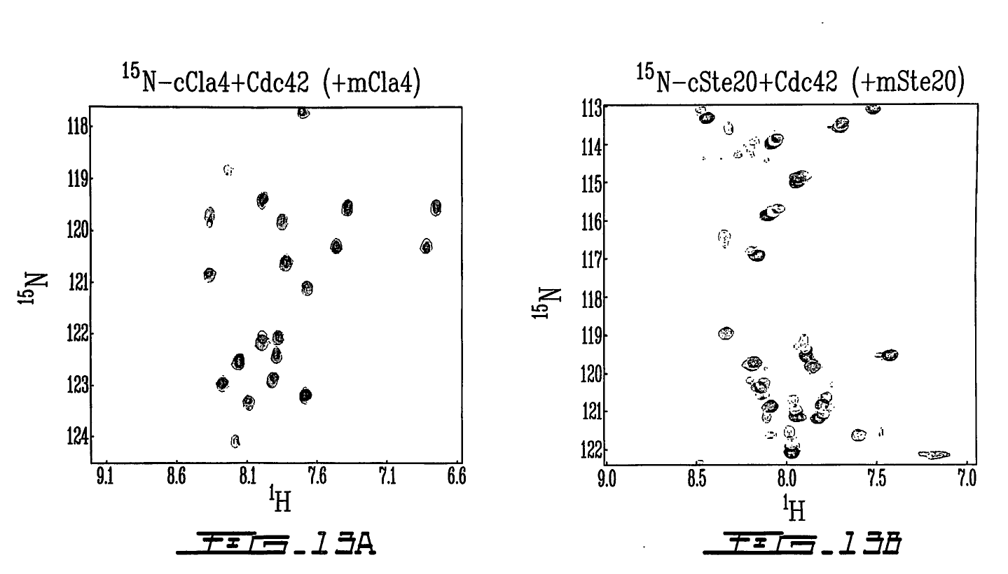

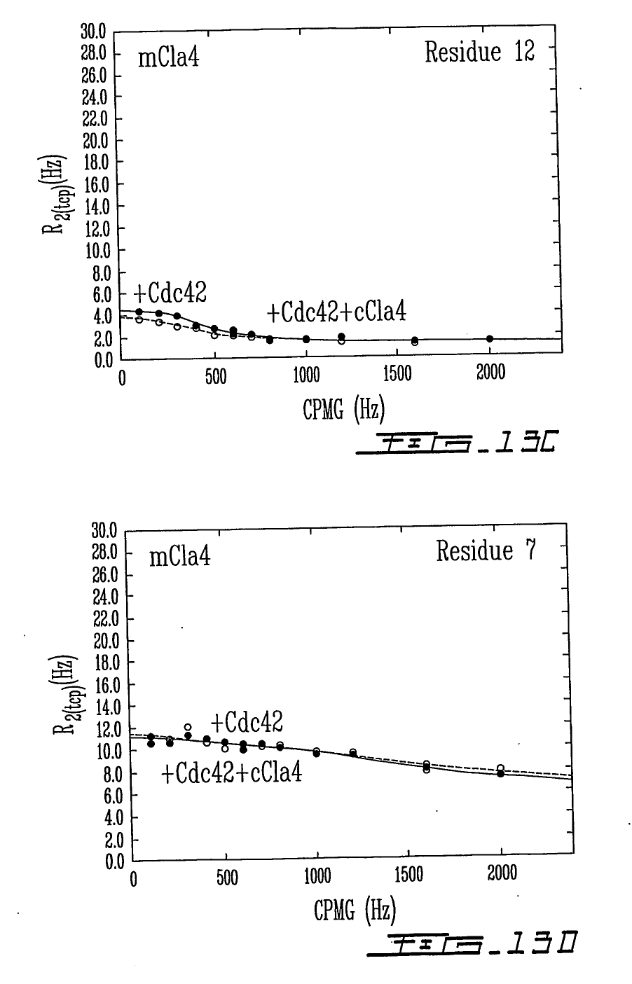

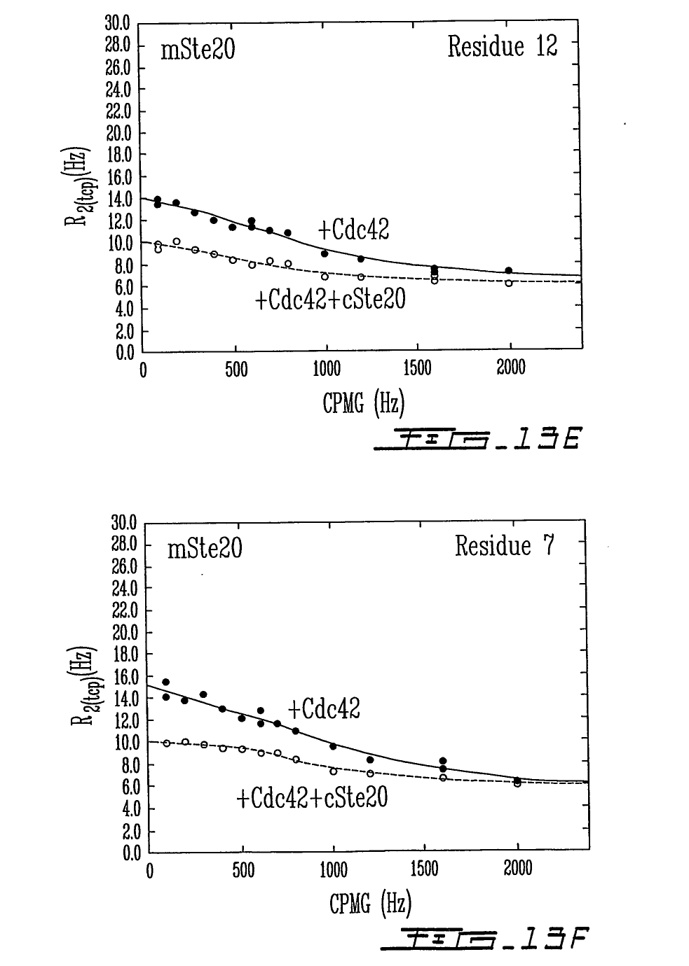

The NMR relaxation dispersion technique can also be used to detect co-operative binding between two peptide ligands as shown in FIGS. 13C to 13E. In this experiment, the dispersion profile for the Cdc42-peptide complexes was monitored with either mCla4 or mSte20 (see FIG. 14 for the identities of these peptides) in the absence and presence of the cognate cCRIB fragments (FIG. 14). Changes in the dispersion profiles indicate perturbation of the binding kinetics at the mCRIB:Cdc42 interface induced by binding at a distal site. The results indicate a co-operative binding to Cdc42 in the Ste20 system but not with the Cla4 peptides (FIGS. 13C to 13E). These results were substantiated by titrating mCRIBs into ¹⁵N-labeled cCRIB saturated with Cdc42 (FIGS. 13A and 13B). However, the advantage of the NMR relaxation dispersion experiments is the possibility to quantitate the dissociation constants in the absence and presence of molecules (such as the cCRIB peptide, FIG. 14) targeting distal binding sites. A decreased off-rate induced by distal binding would constitute the basis for positive cooperativity while increased off-rate would indicate negative cooperativity between the two sites. It was anticipated that these experiments would be critical to identifying and developing a novel class of ligand molecules which have weak affinities when bound separately to the target protein, however, when used in tandem exhibit tight and specific binding via cooperative processes. As well, one can link two or a number of molecules binding to non-overlapping sites on a protein surface to create polyvalent and high-affinity ligand molecules from individually weak-binding ligands (Shuker, S. B., Hajduk, P. J., Meadows, R. P., and Fesik, S. W. (1996) Science 274, 1531-1534; and Kramer, R. H. and Karpen, J. W. (1998) Nature 395, 710-713; and Rao, J., Lahiri, J., Isaacs, L., Weis, R. M., and Whitesides, G. M. (1998) Science 280, 708-711; and Song, J. and Ni, F. (1998) Biochem.Cell Biol. 76, 177-188). Experiments of the type shown in FIGS. 13C to 13E can help the selection of ligands to be used for the linking. An added advantage of the relaxation dispersion method is that it requires small amounts of the target protein, e.g. Cdc42, and is not limited by the size of the binding protein. In addition, many small molecule compounds can be screened at a time.

These experiments illustrated in FIGS. 13A to 13F clearly indicate an increased affinity for Cdc42 induced by addition of the absent peptide for Ste20, suggesting an allosteric mechanism for binding. Such a mechanism was not detected for the Cla4 peptides and implies a binding diversity of these homologous CRIB peptides for Cdc42.

In FIG. 14, the extended CRIB's (eCRIB) comprise the CRIB motif, plus ˜20 residues to the C-terminus and exhibit tight (K_{D}˜0.5 nM) binding to CaCdc42. These sequences were dissected into two fragments: the minimal CRIB (mCRIB), mCla4 and mSte20, and the C-terminal CRIB (cCRIB), cCla4 and cSte20.

### EXAMPLE 6

### Quantitative k_{off} Determination With Ligand Mixtures: ¹⁵N-Labeled Peptides Competing for the Same Binding Site on a Protein

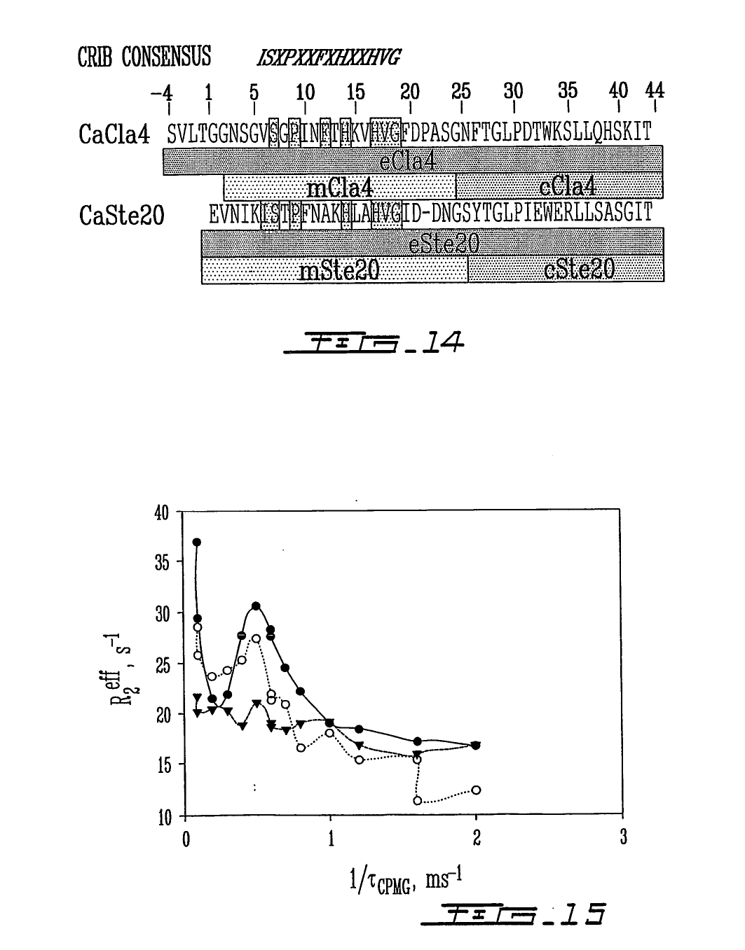

Recombinant expression was used to prepare a mixture of four hirudin-based and uniformly ¹⁵N-labeled peptides, Hir(54-65) (GDFEEIPEEYLQ) (SEQ ID NO:19), HRC2 (GDYEEIPEEYLQH_{L}) (SEQ ID NO:12), HRC3 (GDLEEIPEEYLQH_{L}) (SEQ ID NO:13), and HRC4 (GDGEEIPEEYLQ) (SEQ ID NO:14), where a one-letter code is used to define the amino acid sequence, and H_{L} stands for homoserine lactone. The peptide sequences differ in the third amino acid position and the presence of the homoserine lactone at the C-terminus. The ¹⁵N relaxation dispersion profiles were collected for the peptide mixture in the presence of sub-equimolar amounts of human prothrombin (FIGS. 15A and 15B). The quality of relaxation dispersion profiles was found to be very poor when broadened peaks with noticeable relaxation dispersion overlap with sharp peaks displaying no or very little relaxation dispersion. This situation is especially severe in the case of the mixture of peptides with very similar amino acid sequence, when special protocols are needed for spectral resolution and signal quantitation. It is no longer possible to use peak integrals for quantitating the dispersion, since the contribution from the slowly relaxing and therefore non-informative peaks dominate the total integrated intensity. An alternative for partially overlapped peaks is to measure peak intensities (FIG. 15A). The resulting ¹⁵N relaxation dispersion can be observed qualitatively (FIG. 15B) and, although displaying poor accuracy, follows the same trends as those for the Hir(54-65) peptide in complex with prothrombin (FIG. 8). In FIGS. 15A and 15B, the Hir(54-65) peptide was mixed in 50 mM sodium phosphate buffer, 10% D₂O, at pH 5.5, with three uniformly ¹⁵N-labeled homologous peptides GDYEEIPEEYLQH_{L} (SEQ ID NO:12), GDLEEIPEEYLQH_{L} (SEQ ID NO:13), and GDGEEIPEEYLQ (SEQ ID NO:14), where a one-letter code is used to define the amino acid sequence, and H_{L} stands for homoserine lactone. The curves were obtained by using intensities of the encircled peaks, marked in FIG. 15A). The molar ratio of human prothrombin:peptide is approximately 1:30. The spectra were recorded at a ¹⁵N frequency of 81.076 MHz.

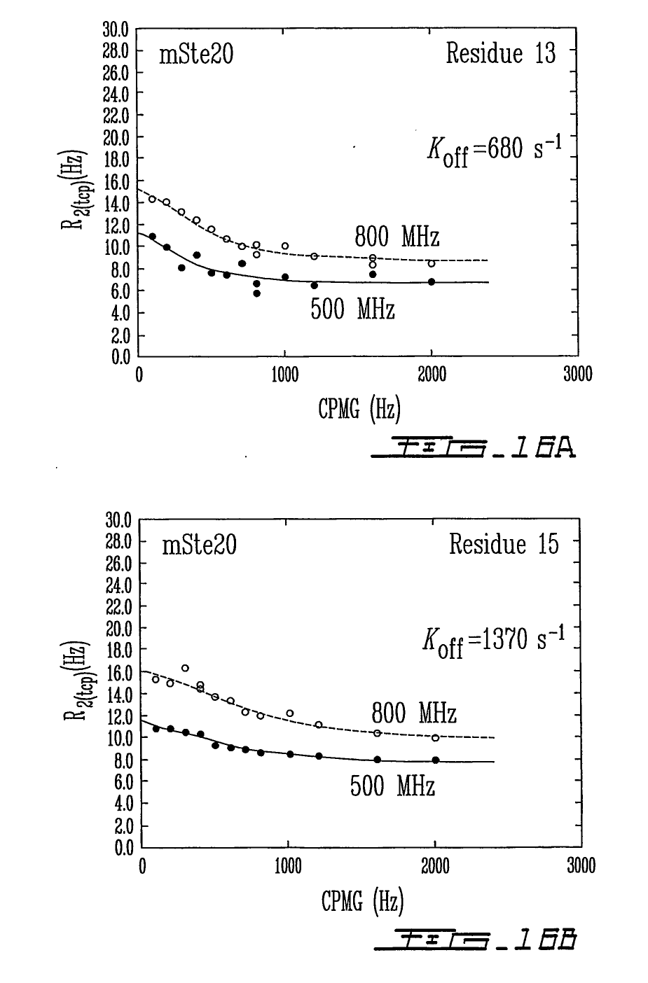

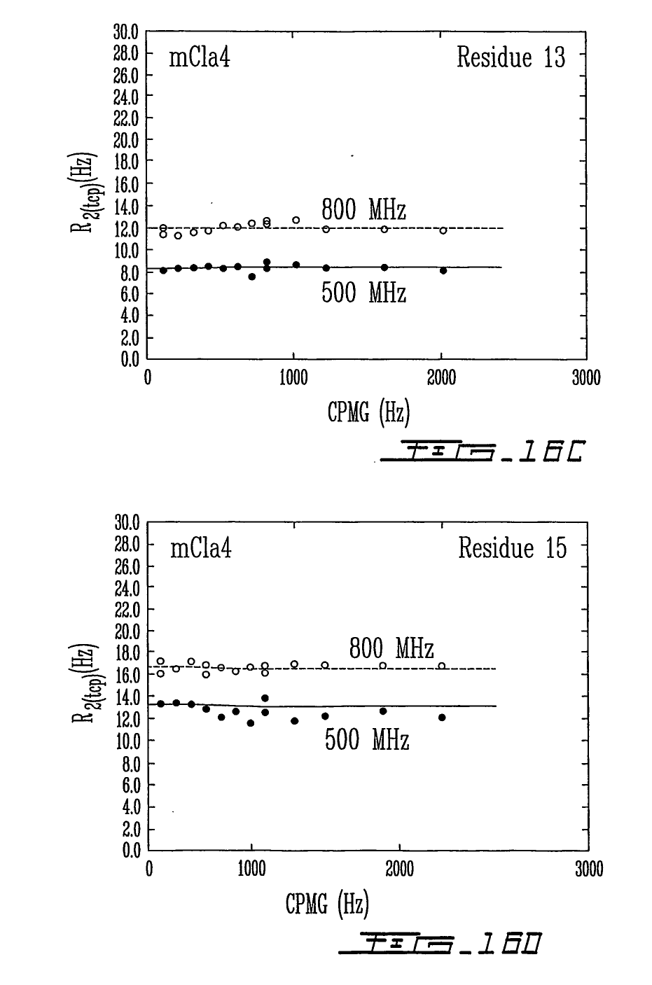

¹⁵N relaxation dispersion curves were collected for the two ¹⁵N labeled CRIB peptides, mSte20 and mCla4 (FIGS. 13A to 13F and 14), mixed together in an approximately equal concentration. The peptide mixture did not show responses to the CPMG pulse rates, indicating lack of binding effects between the two peptides. FIGS. 16A to 16D shows the ¹⁵N relaxation dispersion of two sets of resonance peaks in the peptide mixture, assigned to residues 13 and 15 of the mSte20 and mCla4 peptides, respectively, after the addition of Cdc42. It is seen that only residues of the mSte20 peptide exhibit relaxation dispersion, suggesting a stronger binding affinity for this peptide. The dissociation rate constants for each of the peptides can be quantitated by further titration of the peptide mixture with Cdc42 and collection of ¹⁵N relaxation dispersion curves of the peptide NMR signals at each Cdc42 concentrations (see Example 1). In FIGS. 16A to 16D, Cdc42 was added to ¹⁵N-labeled mSte20 and mCla4 peptides in ˜1:20 molar ratio. The data indicate a CPMG response from the mSte20 peptide (FIGS. 16A and 16B) and not from mCla4 (FIGS. 16C and 16D) suggesting mSte20 has higher affinity for the site on Cdc42 (in agreement with our previous observations). Due to the potentially complicated nature of binding at the Cdc42 surface, quantitative analysis will require a titration series as proposed in Example 1.

### EXAMPLE 7

### Applications to High-Affinity Protein-Protein Interactions: Identification of Binding “Hotspots” Through Peptide Fragmentation

A peptide named FD22, has been discovered as a potent bivalent inhibitor of human thrombin with IC₅₀≈20 nM. FD22 has the sequence of Phe-Asp₄₅-Pro-Arg-Pro-Gln-Ser₅₀-His-Asn-Asp-Gly-Asp₅₅-Phe-Glu-Glu-Ile-Pro₆₀-Glu-Glu-Tyr-Leu-Gln₆₅ (SEQ ID NO:20) and binds to thrombin via both the anion-binding exosite-I and the catalytic active site. The addition of active thrombin to the peptide FD22 (dissolved at a concentration of ˜0.9 mM in 50 mM sodium phosphate buffer, 10% D₂O, 0.2 mM EDTA, at pH 5.5), caused slow and specific proteolytic cleavage at the Arg₄₇-Pro₄₈ peptide bond (taking >60 hours for the cleavage to complete). Upon completion of the cleavage, the sample contains a mixture of two peptides, Phe-Asp₄₅-Pro-Arg (FD22-N) (SEQ ID NO:17) and Pro-Gln-Ser₅₀-His-Asn-Asp-Gly-Asp₅₅-Phe-Glu-Glu-Ile-Pro₆₀-Glu-Glu-Tyr-Leu-Gln₆₅ (FD22-C) (SEQ ID NO:18) that should bind separately to the active site and the anion-binding exosite I of thrombin. Residues Phe₅₆, Glu₅₇, Glu₅₈, Ile₅₉, Tyr₆₃, Leu₆₄ and Gln₆₅ of the 55-65 region of the FD22-C fragment displayed pronounced resonance line broadening and ¹⁵N NMR relaxation dispersion (FIGS. 12A to 12H), while residues Gly₅₄-Asp₅₅ had slowly relaxing ¹⁵N signals and lack of relaxation dispersion. These data indicate that residues Glu₅₆-Gln₆₅ constitute a binding hotspot for the high affinity full-length FD22 peptide, in agreement with previous findings (Ni, F., Konishi, Y., and Scheraga, H. A. (1990) Biochemistry 29, 4479-4489). In addition, specific relaxation dispersion of the backbone ¹⁵N atom of Arg₄₇ (FIGS. 12A to 12H) shows that the small tetrapeptide Phe-Asp₄₅-Pro-Arg (or FDPR) (SEQ ID NO:17) may bind to thrombin with a decreased off-rate in the presence of the FD22-C peptide in contrast to other related pentapeptides, GLDPR (SEQ ID NO:7), GVDPR (SEQ ID NO:8), GFNPR (SEQ ID NO:9), GPNPR (SEQ ID NO:10), GFSAR (SEQ ID NO:11), and GVSPR (SEQ ID NO:6) (see Example 4).

The results obtained with the FD22 peptide indicate that the present techniques can also provide binding information for tight and essentially irreversible protein-protein complexes. This is exquisitely illustrated for the complexes of the Cdc42 protein with peptide fragments derived from two signaling kinases (FIGS. 13A to 13F and 14). Cdc42 binds tightly with the 40-residue CRIB domains of Candida Cla4 and Ste20 (K_{D}˜0.5 nM). When subjected to the relaxation dispersion techniques, these complexes exhibit no response, as expected for a tight binding complex. The full-length CRIB domains were dissected into two peptide fragments (FIG. 14). When subjected to the relaxation dispersion analysis the peptide fragments exhibit different binding preferences for Cdc42 (outlined below), which is unlikely to be determined from normal NMR or other analyses of the full-length peptides. ¹⁵N relaxation dispersion spectroscopy coupled with peptide fragmentation can therefore be used for the identification of binding ‘hotspots’ for complexes involving Cdc42 and the two CRIB (Cdc42/Rac interactive binding) proteins, Ste20 and Cla4 from Candida albicans.

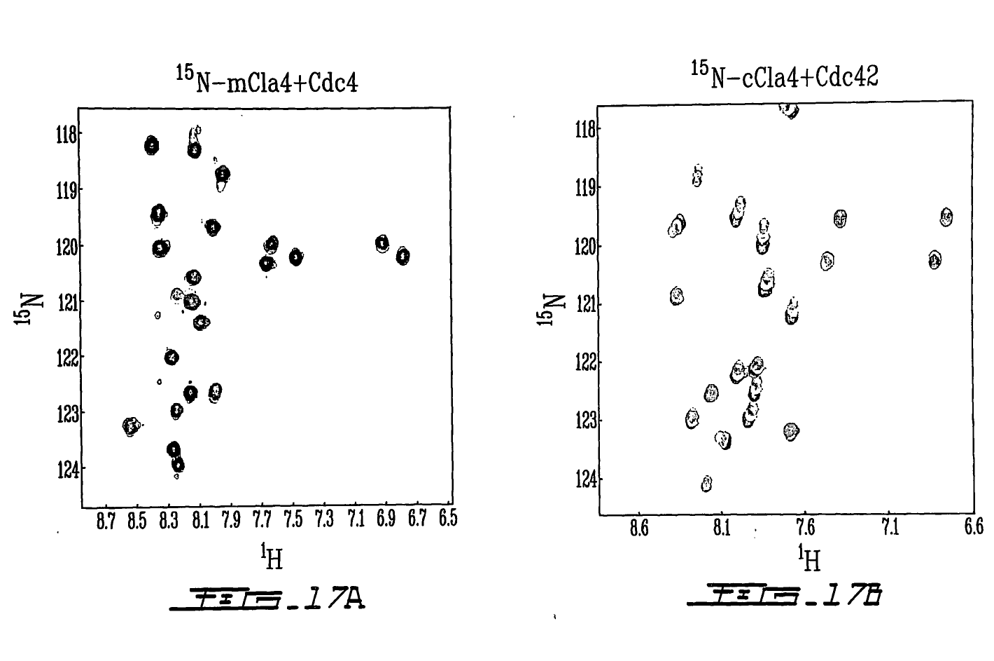

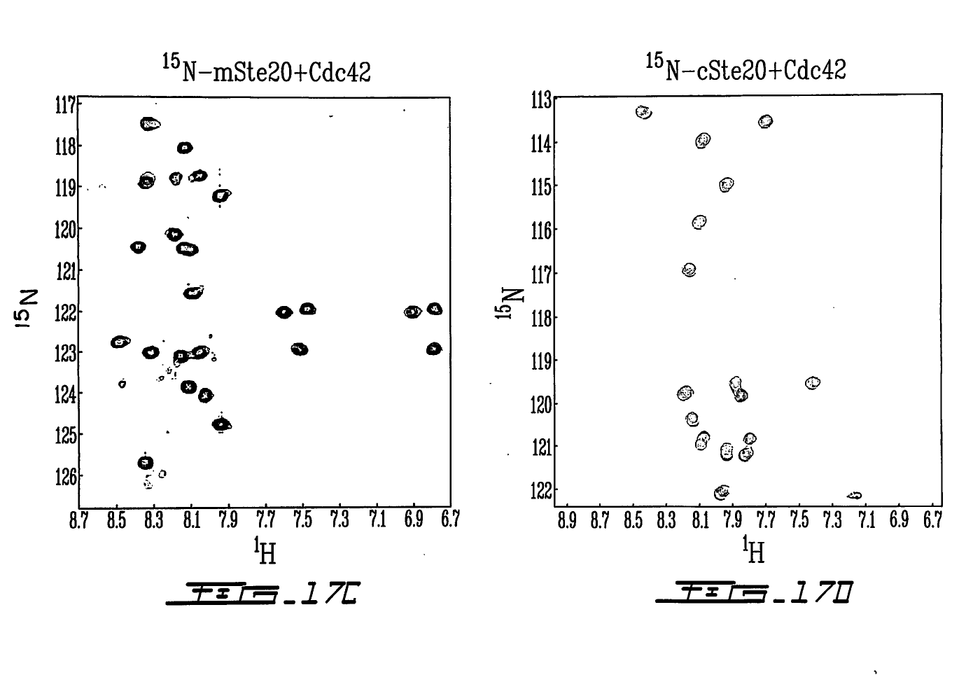

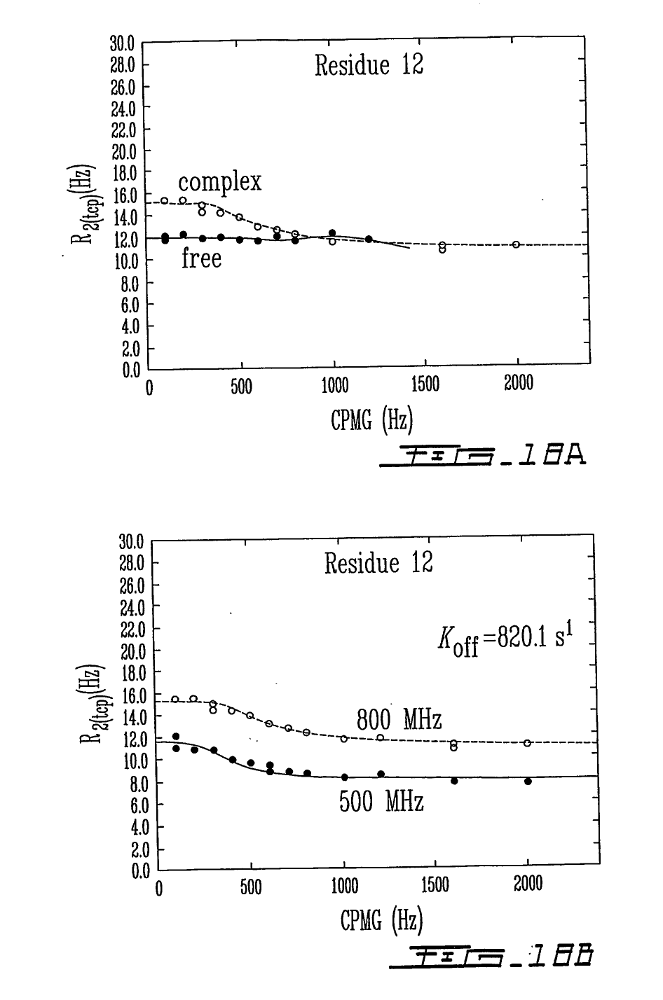

For these experiments, two peptide fragments of the extended CRIB region from the Cla4 kinase (FIG. 14): (i) mCla4 (22 residues) including the CRIB motif, and (ii) cCla4 which comprises residues directly to the C-terminus of the CRIB motif were over-expressed. FIGS. 17A and 17B shows the perturbations to the ¹H—¹⁵N HSQC spectra of ¹⁵N-labeled mCla4 (FIG. 17A) and ¹⁵N-labeled cCla4 (FIG. 17B) upon addition of unlabeled Cdc42. The cCla4 spectrum undergoes minor peaks shifts indicating that the cCla4-Cdc42 complex may be in the fast-exchange regime, hence weak binding. In contrast, the mCla4 peptide may bind tighter to Cdc42, as shown by the extensive broadening and eventual disappearance of resonances. Different results were obtained when titration experiments were performed on the analogous peptide fragments from Ste20: mSte20 and cSte20 (FIG. 14). The cSte20 peptide showed no visible interaction with Cdc42 (FIG. 17D), whereas the HSQC signals of the mSte20 fragment broaden more easily than the analogous mCla4 peptide (FIG. 17C), suggesting that mSte20 may bind tighter or in a different mode to mCla4. In FIGS. 17A to 17D, identical protein and peptide concentrations were in all titrations. Spectra are colored from black (free peptide) to red (Cdc42 in 10-fold excess). Differential binding properties between the two kinase fragments are seen.

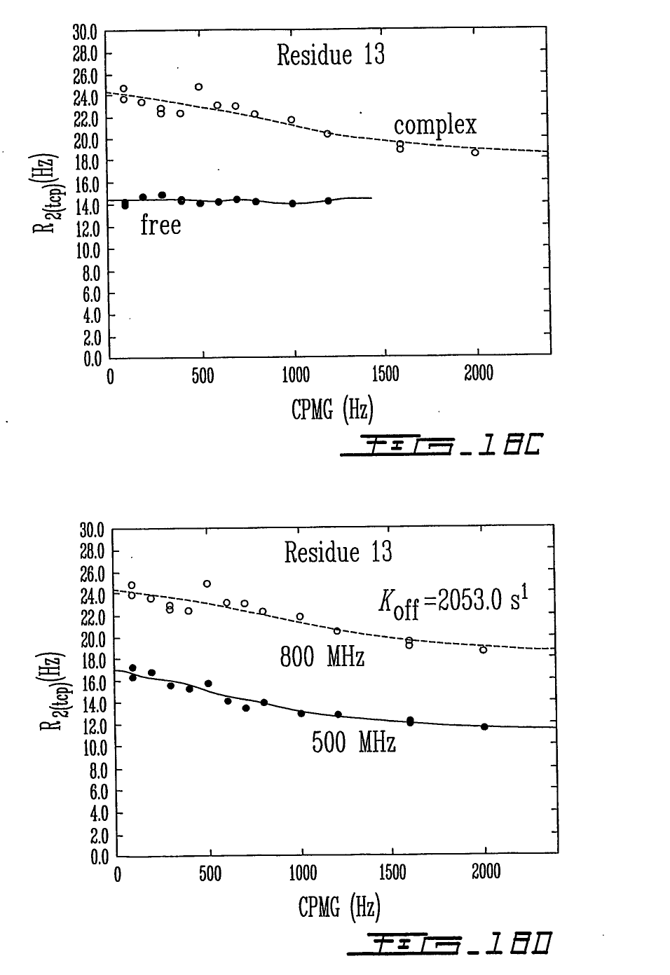

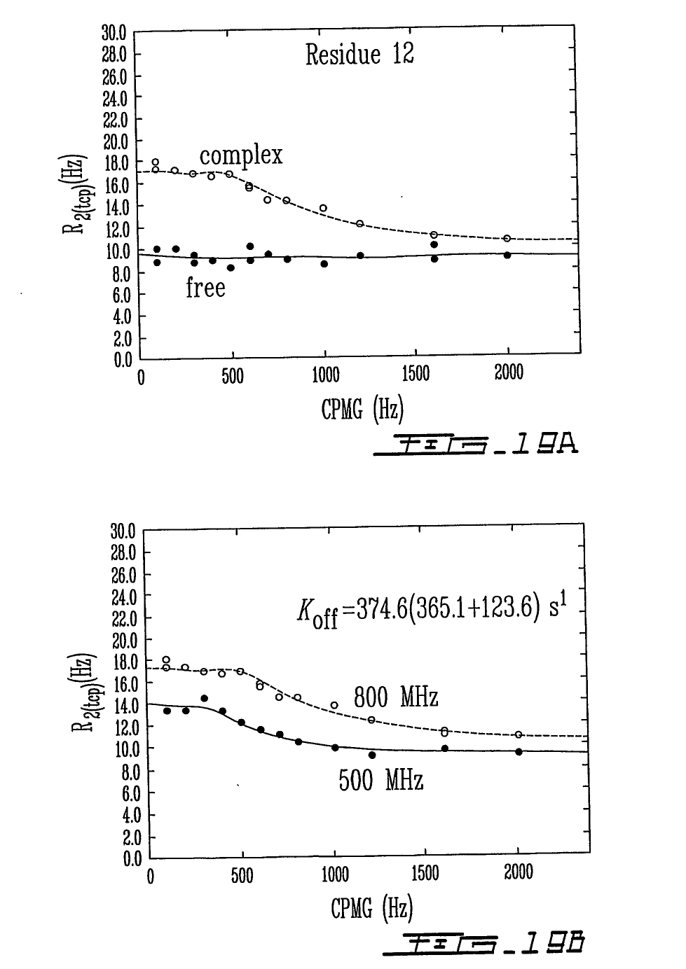

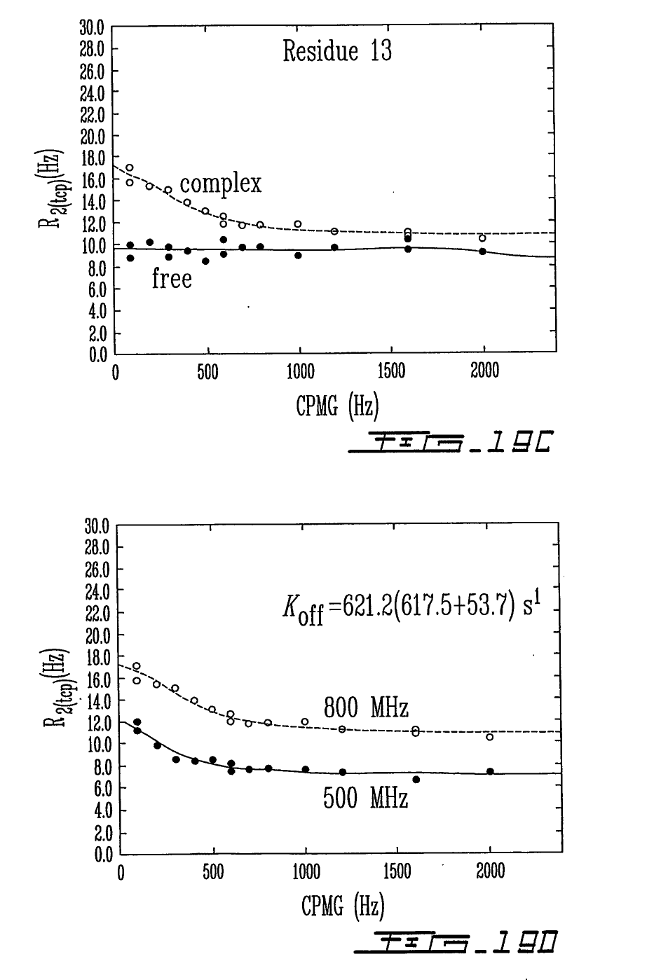

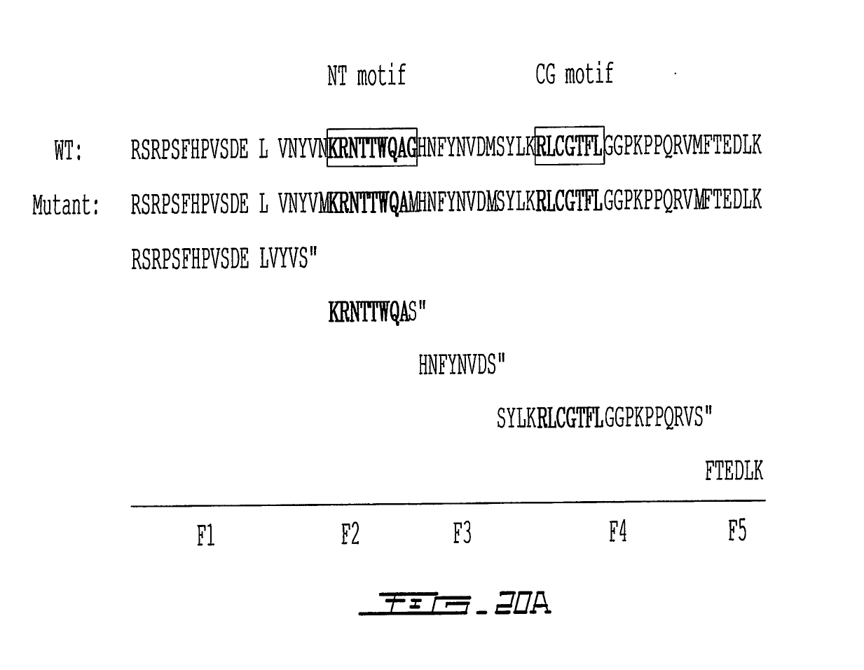

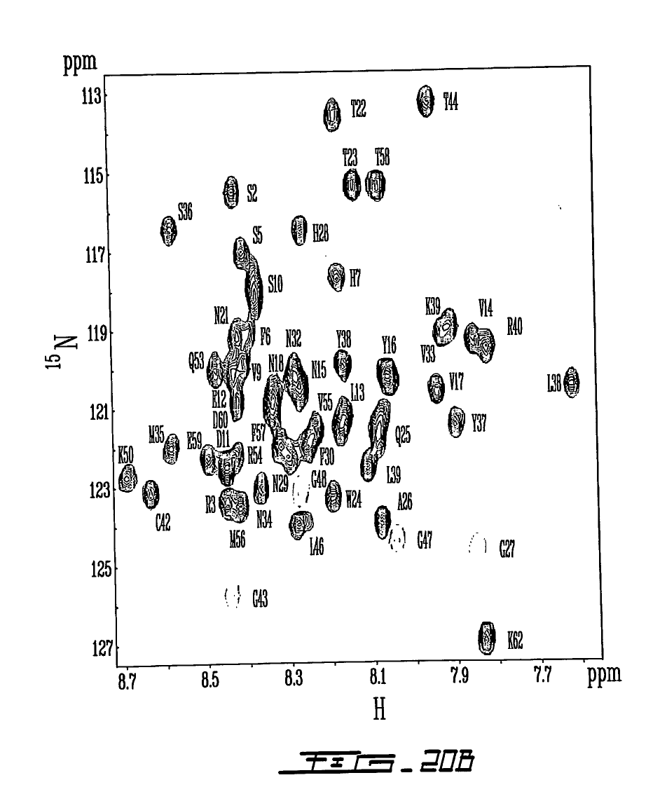

¹⁵N relaxation dispersion spectroscopy was then used to probe the kinetics (specifically the off-rates), of the transient mCRIB complexes with Cdc42 in order to rank the relative affinities of similar peptide fragments. FIGS. 18A to 18D and 19A to 19D show representative curves for selected residues of the mSte20 and mCla4 peptides in complex with Cdc42. The free mSte20 and mCla4 peptides show no response to the CPMG pulses (FIGS. 18A and 19A red datasets), whereas addition of Cdc42 in ˜ 1/10^{th} the concentration of the peptide induces significant responses, indicating the formation of a transient complex (black dataset). Acquiring data at two magnetic field strengths can potentially yield more accurate off values (FIGS. 18B and 19B). Unfortunately, the simple two-state binding model employed may not be applicable to all residues in this system. However, it is noted that the fits do indicate generally lower off-rates for mSte20 (FIGS. 18B, and 19B), in agreement with titration data.

In FIGS. 18A to 18D and 19A to 19D, apparent values for k_{off}, based on a two-site exchange model are shown.

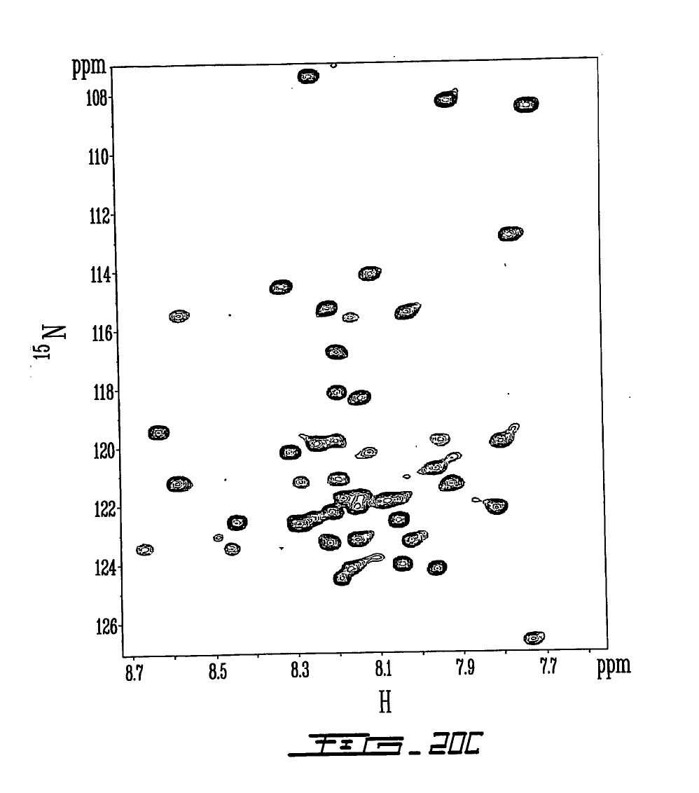

A large and tight-binding protein or peptide can also be fragmented into more than two subfragments for use in NMR relaxation dispersion studies. This is illustrated with the propeptide of human cathepsin B (FIG. 20A), which is a 62 amino acid protein fragment binding tightly to cathepsin B (K_{I}=0.4 nM at pH 6.0 (Fox, T., de Miguel, E., Mort, J. S., and Storer, A. C. (1992) Biochemistry 31, 12571-12576)). The propeptide has been found to contain two important binding motifs, labeled as the NT motif and the CG motif (Chen, Y., Plouffe, C., Menard, R., and Storer, A. C. (1996) FEBS Lett. 393, 24-26). Methionine residues were inserted into the propeptide sequence by site-directed mutagenesis in order to prepare a mixture of essentially equimolar concentrations of the individual peptide fragments F1, F2, F3, F4, and F5 (FIG. 20A). FIG. 20B shows the [¹H—¹⁵N]—HSQC spectrum of the full-length propeptide, which was assigned through NMR experiments including 3D [¹H—¹⁵N]—HSQC-TOCSY and [¹H—¹⁵N]—HSQC-NOESY. The [¹H—¹⁵N]—HSQC peaks of the fragmented propeptide are much sharper (FIG. 20C), and can be utilized for ¹⁵N relaxation dispersion experiments.

In FIGS. 20B and 20C, the spectra were recorded in 50 mM sodium acetate-d₃ buffer, pH 5.5, at 500 MHz, and at 288 K.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method to identify a ligand site obeying a two-state or more complex binding behavior in a transient complex of a ligand with a target molecule, said method comprising the steps of:
a) preparing a ligand with at least one nucleus detectable by NMR;
b) collecting NMR relaxation dispersion profiles for said nucleus at two or more magnetic fields;
c) determining apparent transverse relaxation rates from said dispersion profiles of step b);
d) assigning resonance peaks to said nucleus of the ligand with one- and/or multi-dimensional NMR;
e) contacting the ligand of step a) with at least one concentration of a target molecule;
f) for each contacting of said ligand with at least one concentration of said target molecule as defined in step
e) collecting NMR relaxation dispersion profiles for said ligand contacted with said at least one concentration of said target molecule for every concentration of said target molecule at two or more magnetic fields;
g) fitting said dispersion profiles obtained in step f) by including the relaxation rates of step c) and using a two-state exchange model independently for every nucleus, and independently or simultaneously for every concentration of the target molecule; and
h) determining a ligand site obeying a two-state binding behavior based on feasibility of extracted R_{2b} and p_{b} parameters obtained by the fitting of step g), wherein R_{2b} represents a nominal transverse relaxation rate of said ligand bound to the target molecule and p_{b} represents a nominal fraction of the bound ligand.

2. The method of claim 1, wherein the ligand of step a) has at least two detectable nuclei.

3. The method of claim 1 or 2, wherein the ligand in step e) is contacted with at least two concentrations of a target molecule.

4. The method of claim 3, wherein the ligand is contacted with three concentrations of target molecules.

5. A method to determine quantitatively the dissociation rate constant (k_{off}) for a transient complex of a ligand with the target molecule comprising the steps of:
a) Identifying a ligand site obeying a two-state binding behavior in a transient complex of a ligand with a target molecule with the method as defined in claim 1, 2, 3 or 4; and
b) Extracting k_{off} values for the ligand sites obeying two-site exchange mechanism, said off values being a measure of the affinity of a transient complex of the ligand with the target molecule.

6. A method according to claim 1, 2, 3, 4, or 5, wherein the ligand is a polypeptide.

7. A method according to claim 1, 2, 3, 4, 5 or 6, wherein the ligand is a ¹⁵N-enriched polypeptide.

8. A method according to claim 1, 2, 3, 4, 5, 6 or 7, wherein the ligand is a mixture of polypeptides and/or molecules.

9. A method according to claim 1, 2, 3, 4, 5, 6, 7 or 8, wherein the target molecule is a protein or a protein assembly.

10. Use of the method as defined in claim 1, 2, 3, 4, 5, 6, 7, 8 or 9 to determine amino acid residues with detectable NMR relaxation dispersion as constituting binding hot-spots.

11. Use of the method as defined in claim 1, 2, 3, 4, 5, 6, 7, 8, 9 or 10 to identify two or more ligands that can be linked together to create high-affinity molecules.

12. Use of the method of claim 1, 2, 3, 4, 5, 6 7, 8 or 9 to study high-affinity protein-protein interactions or slow-dissociating ligand-target complexes.

13. The use of claim 10, 11 or 12, wherein the ligand is a polypeptide or a protein.

14. The use of claim 10, 11, 12 or 13, wherein the ligand is ¹⁵N-enriched.

15. The method of any one of claims 1 to 9, where the NMR relaxation dispersion profiles are collected by a CPMG method.

16. The use of claims 10 to 14 where the NMR relaxation dispersion profiles are collected by a CPMG method.
